(19) United States
(12) Patent Application Publication
(10) Pub. No.: US 2021/0171908 A1
(43) Pub. Date: Jun. 10, 2021

(54) ALLOGENEIC T-CELLS AND METHODS FOR PRODUCTION THEREOF

(71) Applicants: LONZA WALKERSVILLE, INC., Walkersville, MD (US); LONZA ISRAEL LTD., Haifa (IL)

(72) Inventors: Yonatan LEVINSON, Walkersville, MD (US); Senthil RAMASWAMY, Walkersville, MD (US); Alex SARGENT, Walkersville, MD (US); Ariel GILERT, Haifa (IL); Alona FELDMAN, Haifa (IL); Frida GOTLIEB, Haifa (IL)

(21) Appl. No.: 17/088,759

(22) Filed: Nov. 4, 2020

Related U.S. Application Data
(60) Provisional application No. 62/930,617, filed on Nov. 5, 2019.

(51) Int. Cl. C12N 5/0783; C12N 15/90

(52) U.S. Cl. CPC C12N 5/0636; C12N 2310/20; C12N 2800/80; C12N 15/907

(57) ABSTRACT

The present invention provides methods for producing allogeneic T-cells, including the use of an engineered nuclease under the control of a controllable promoter. By preparing T-cells with an inducible nuclease, large volumes of cells can be prepared, each of which contains the ability to individually produce the desired nuclease. These cells can then be modified as desired through the introduction of a gene of interest, or an undesired gene can be knocked-out. Also provided herein are allogeneic T-cells for use in various therapeutic applications.

### CROSS-REFERENCE TO RELATED APPLICATIONS

The present application claims benefit of U.S. Provisional Patent Application No. 62/930,617, filed Nov. 5, 2019, the disclosure of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 15, 2020, is named 0132-0081US1 SL.txt and is 1,504 bytes in size.

### FIELD OF THE INVENTION

The present invention provides methods for producing allogeneic T-cells, including the use of an engineered nuclease under the control of a controllable promoter. By preparing T-cells with an inducible nuclease, large volumes of cells can be prepared, each of which contains the ability to individually produce the desired nuclease. These cells can then be modified as desired through the introduction of a gene of interest, or an undesired gene can be knocked-out. Also provided herein are allogeneic T-cells for use in various therapeutic applications.

### BACKGROUND OF THE INVENTION

As clinical adoption of advanced cell therapies begins to gain traction, more attention is turning to the underlying manufacturing strategies that will allow these therapies to benefit patients worldwide. Successful results from immunotherapy trials using chimeric antigen receptor (CAR) T-Cells provide new hope to patients suffering from previously untreatable cancers. While cell therapies hold great promise clinically, high manufacturing costs relative to reimbursement present a formidable roadblock to commercialization.

One of the challenges facing CAR T-Cell therapies is the generation of allogenic cells that can be used for any patient. Scale up of allogeneic T-cell therapies can be prohibitively expensive and require long lead times due to the need for high quantities of viral vectors and/or recombinant endonucleases.

What are needed to overcome these challenges are methods for producing T-cell lines that do not require a significant investment in time and money, yet still provide a cell line that can be used for multiple patient populations. The present invention fulfills these needs.

### SUMMARY OF THE INVENTION

In some embodiments, provided herein is a method of producing a T-cell line for use in an allogeneic application, comprising: introducing a nucleic acid molecule encoding an engineered nuclease under the control of a controllable promoter into a T-cell line; integrating the nucleic acid molecule into the genome of the T-cell line; and expanding the T-cell line.

In further embodiments, provided is a method of producing a genetically modified T-cell line, comprising: introducing a nucleic acid molecule encoding a CRISPR-associated nuclease under the control of a controllable promoter into a T-cell line; integrating the nucleic acid molecule into the genome of the T-cell line; expanding the T-cell line; inducing expression of the CRISPR-associated nuclease by activating the controllable promoter; introducing a guide-RNA and a gene of interest into the expanded T-cell line; knocking out expression of a T-cell receptor and introducing the gene of interest into the genome of the T-cell line; and recovering the genetically modified T-cell line.

Also provided herein is a method of producing a chimeric antigen receptor (CAR) T-cell line, comprising: introducing a nucleic acid molecule encoding a Cas9 nuclease under the control of a controllable promoter into a T-cell line; integrating the nucleic acid molecule into the genome of the T-cell line; expanding the T-cell line; inducing expression of the Cas9 nuclease by activating the controllable promoter; introducing a guide-RNA and a nucleic acid encoding a chimeric antigen receptor (CAR) into the expanded T-cell line; knocking out expression of a T-cell receptor and introducing the nucleic acid encoding the CAR into the genome of the T-cell line; and recovering the CAR T-cell line.

In additional embodiments, provided herein is an allogeneic T-cell line, comprising a CRISPR-associated (Cas) nuclease under the control of a controllable promoter integrated into the genome of the T-cell line.

### BRIEF DESCRIPTION OF THE DRAWINGS

FIG. 1 shows a schematic representation of both autologous and allogeneic approaches to T-cell therapies.

FIG. 2 shows steps in the production of allogeneic T-cells.

FIG. 3 shows three exemplary phases of allogeneic T-cell production.

FIGS. 4A-4B show an exemplary derepressible promoter system for use herein.

FIGS. 4C-4D shows an inducible vector system for use in embodiments hereof.

FIG. 4E shows the TRE3G Tet-On system for use in embodiments hereof.

FIGS. 5A-C show three treatment protocols for transduction of T-cells with a Cas9 inducible vector.

FIGS. 6A-6B show the results of transduction of T-cells with a Cas9 inducible vector. See symbols on legend to track line-graphs.

FIGS. 7A-7B shows the number of viable cells after selection, and during cell expansion.

FIG. 8 shows measurement of exhaustion makers, senescence markers, activation markers, and T-cell markers for the three treatment protocols.

FIG. 9 shows the results of induction of Cas9 expression in T-cells.

FIGS. 10A and 10B show T-cell expansion after cryopreservation.

FIG. 11 shows three approaches for TRAC gene knockout.

FIGS. 12A-1 through 12C show TRAC knock-out 4 days post nucleofection.

FIGS. 13A-1 through 13C show TRAC knock-out 7 days post nucleofection.

FIGS. 14A-1 through 14C show TRAC knock-out 14 days post nucleofection.

FIGS. 15A-15B show a compilation of knock-out experiments.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host T-Cells, expression vectors, and/or composition of the invention. Furthermore, compositions, systems, cells, and/or nucleic acids of the invention can be used to achieve any of the methods as described herein.

### Chimeric Antigen Receptor T-Cells

A chimeric antigen receptor T-cell, or “CAR T-cell,” is a T-cell (also called T Cell herein) that is modified with a chimeric antigen receptor (CAR) to more specifically target cancer cells. In general, a CAR includes three parts: the ectodomain, the transmembrane domain, and the endodomain. The ectodomain is the region of the receptor that is exposed to extracellular fluid and includes three parts: a signaling peptide, an antigen recognition region, and a spacer. The signaling peptide directs the nascent protein into the endoplasmic reticulum. In CAR, the signaling peptide is a single-chain variable fragment (scFv). The scFv includes the variable fragments of the light chain, connected with a short linker peptide. In some embodiments, the linker includes glycine and serine. In some embodiments, the linker includes glutamate and lysine.

The transmembrane domain of the CAR is a hydrophobic α-helix that spans the membrane. In some embodiments, the transmembrane domain of a CAR is a CD28 transmembrane domain. In some embodiments, the CD28 transmembrane domain results in a highly expressed CAR. In some embodiments, the transmembrane domain of a CAR is a CD3-ζ transmembrane domain. In some embodiments, the CD3-ζ transmembrane domain results in a CAR that is incorporated into a native T-cell receptor.

The endodomain of the CAR is generally considered the “functional” end of the receptor. After antigen recognition by the antigen recognition region of the ectodomain, the CARs cluster, and a signal is transmitted to the cell. In some embodiments, the endodomain is a CD3-endodomain, which includes 3 immunoreceptor tyrosine-based activation motifs (ITAMs). In this case, the ITAMs transmit an activation signal to the T-cell after antigen binding, triggering a T-cell immune response. Additional CAR designs known in the art can also be utilized in the practice of the methods described herein.

During production of CAR T-cells, T-cells are removed from a human subject, genetically altered, and re-introduced into a patient to attack the cancer cells. CAR T-Cells can be derived from either the patient's own blood (autologous), or derived from another healthy donor (allogenic). In general, CAR T-cells are developed to be specific to an antigen that is overexpressed on a tumor relative to healthy cells.

### Methods for Producing T-Cells for Use in Allogenic Applications

While autologous T-cells represent significant opportunities for treatment of various diseases and in particular cancers, the use of allogenic cells, and in particular methods for producing a line of cells that can be modified as desired for each individual patient represents a tremendous advance for T-cell based therapies. By producing a line of T-cells that can be used for any patient, not only does the supply of potential cells increase dramatically, but so does the cost of producing such cells.

Traditional methods for producing allogeneic T-cells, after removal of the cells from a patient donor, include the step of expanding the cells prior to introduction of the desired CAR for the requisite therapy. However, in such methods, since the T-cell receptor is required for expansion, but must be removed prior to introduction into a patient, all of the CAR introduction must occur after cell expansion. This not only requires a significant amount of virus and desired CAR, but also a significant amount of any engineered nuclease that may be required to perform the genetic editing. This significantly increases cost as well as complexity.

The methods described herein, however, allow for the production of a T-cell that includes within the cell an engineered nuclease under the control of a controllable promoter, but still retain the natural T-cell receptor allowing for expansion. Once the cell is expanded, a desired CAR can then be inserted, the T-cell receptor removed, and the cells take for further processing and suitably for injection into a patient.

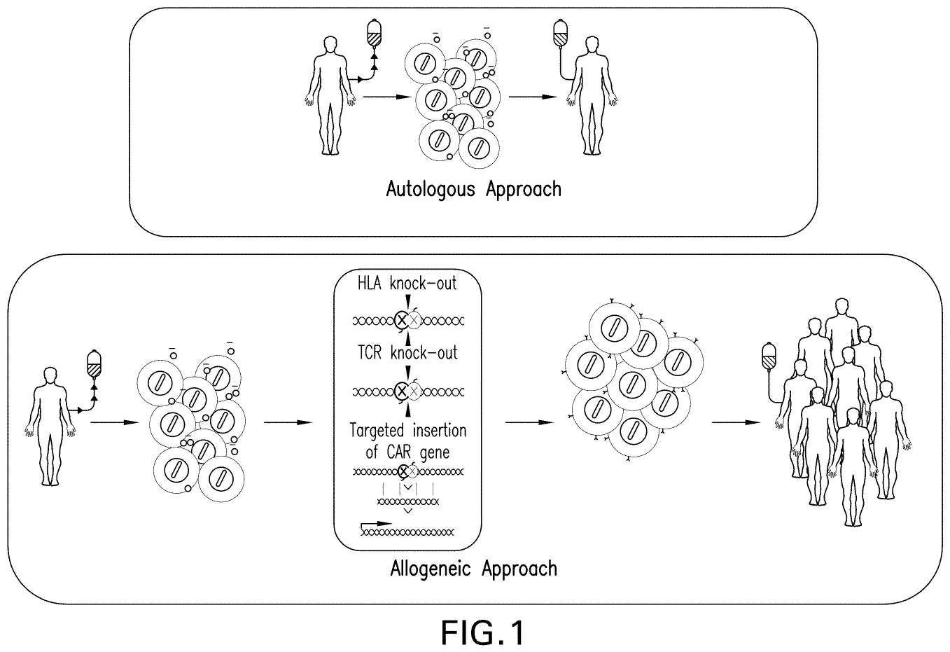

FIG. 1 shows a schematic representation of both autologous and allogeneic approaches to T-cell therapies. In autologous applications, T-cells are isolated from the patient, the CAR construct is virally transduced into a patient's T-cells, and the CAR T-cells are then introduced back into the same patient. In an allogeneic approach, T-cells are isolated from healthy donors, the cells are virally transduced with the desired CAR construct. At the same time, the T-cell receptor is knocked out to prevent graft vs. host disease (GvHD), a prerequisite for universal CAR T therapy (additional genes can also be knocked-out, including B2M and PD1 to help prevent GvHD). The allogeneic T-cells, that include the desired CAR, can now be introduced into any patient. As described herein, producing a T-cell source that can be expanded prior to introduction of the CAR construct would allow for significant increases in scale-up, and also reduce needed resources and costs.

In embodiments then, provided herein is method of producing a T-cell line for use in an allogeneic application. As used herein a “T-cell line” refers to lymphocyte cells developed in the thymus gland and that include a T-cell receptor on their surface. T-cells include immortalized T-cells. As used herein “allogeneic” or “allogenic application” refers to the use of cells from one or more donor sources (often a healthy donor) in therapeutic applications to one or more patients, which can be unrelated to the donor source(s).

In embodiments, the methods described herein include introducing a nucleic acid molecule encoding an engineered nuclease under the control of a controllable promoter into a T-cell line. Methods of introducing a nucleic acid molecule into a T-cell line include the use of various transduction or transfection systems, including various viral systems, for example, the nucleic acid molecule can be introduced into the T-cell line using a lentiviral vector. Additional transduction or transfection systems include nucleofection, use of exosome systems, use of liposome systems, use of polymeric-based systems, etc.

As used herein, “nucleic acid,” “nucleic acid molecule,” or “oligonucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA. RNA includes, but is not limited to, mRNA, tRNA, rRNA, snRNA, microRNA, miRNA, or MIRNA. Nucleic acid also includes RNA that is introduced into a cell, and then is reverse transcribed to DNA, prior to being integrated into the genome of a call.

A “gene” as used herein refers to an assembly of nucleotides that encode a polypeptide, and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence. In some embodiments, genes are integrated with multiple copies. In some embodiments, genes are integrated at predefined copy numbers.

“Transfection” as used herein means the introduction of an exogenous nucleic acid molecule, including a vector, into a cell. A “transfected” cell comprises an exogenous nucleic acid molecule inside the cell and a “transformed” cell is one in which the exogenous nucleic acid molecule within the cell induces a phenotypic change in the cell. The transfected nucleic acid molecule can be introduced into the cell as RNA, reverse transcribed by the cell into DNA, and then integrated into the host T-Cell's genomic DNA and/or can be maintained by the cell, temporarily or for a prolonged period of time, extra-chromosomally. Host T-Cells or organisms that express exogenous nucleic acid molecules or fragments are referred to as “recombinant,” “transformed,” or “transgenic” organisms. A number of transfection techniques are generally known in the art. See, e.g., Graham et al., Virology, 52:456 (1973); Sambrook et al., Molecular Cloning, a laboratory manual, Cold Spring Harbor Laboratories, New York (1989); Davis et al., Basic Methods in Molecular Biology, Elsevier (1986); and Chu et al., Gene 13:197 (1981). Suitably, transfection of a T-cell with one or more of the vectors described herein utilizes a transfection agent, such as polyethylenimine (PEI) or other suitable agent, including various lipids and polymers, to integrate the nucleic acids into the host T-Cell's genomic DNA. In some embodiments, the transfection includes viral infection (also termed “transduction”), transposons, mRNA transfection, electroporation, or combinations thereof. In some embodiments, the transfection includes electroporation. In additional embodiments, the transfection includes viral transduction. The vector may be a viral vector, such as, for example, a lentiviral vector, a gammaretroviral vector, an adeno-associated viral vector, or an adenoviral vector. In embodiments, the transfection includes introducing a viral vector into the activated T cells of the cell culture. In additional embodiments, the vector is delivered as a viral particle.

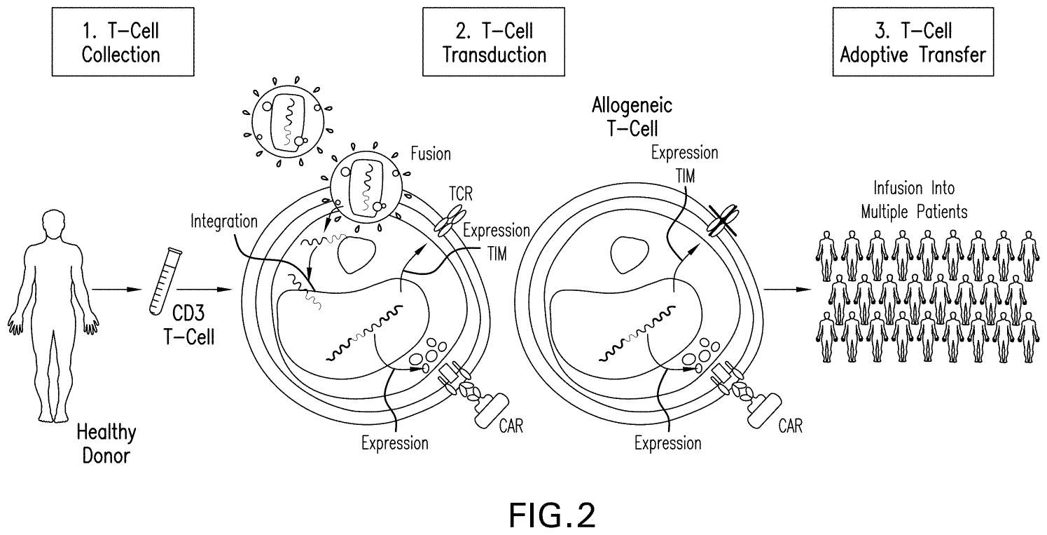

As shown in FIG. 2, T-cells are suitably contacted with a nucleic acid molecule contained within a viral particle to allow for the nucleic acid to integrate into the genome of the T-cell line. The nucleic acid is introduced as RNA, is reverse transcribed by the cell to DNA, and is then integrated into the genome of the cell. This provides a T-cell line that includes within each cell, a genomically integrated engineered nuclease, under the control of a controllable promoter. These T-cells can then be expanded as described herein to produce the T-cell line for use in an allogeneic application.

As used herein, the term “engineered nuclease” refers to a nuclease that has been separated, modified, mutated, and/or altered from it's natural state as a nuclease. A “nuclease” refers to an enzyme that is able to cut a DNA and/or RNA molecule. By engineering the nuclease, the specific location of the cut can be designed and tailored to the desired cell type and/or gene of interest.

Exemplary engineered nucleases that can be inserted into the T-cells include, for example, a meganuclease, a methyltransferase a zinc finger nuclease, a transcription activator-like effector-based nuclease (TALENS), a FokI nuclease, and a CRISPR-associated nuclease. In general, engineered nucleases use a DNA-binding protein which has both a desired catalytic activity and the ability to bind the desired target sequence through a protein-nucleic-acid interaction in a manner similar to restriction enzymes. Examples include meganucleases which are naturally occurring or engineered rare sequence cutting enzymes, zinc finger nucleases (ZFNs) or transcription activator-like nucleases (TALENs) which contain the FokI catalytic nuclease subunit linked to a modified DNA binding domain and can cut one predetermined sequence each. In ZFNs the binding domain is comprised of chains of amino-acids folding into customized zinc finger domains. In TALENs, similarly, 34 amino acid repeats originating from transcription factors fold into a huge DNA-binding domain. In the event of gene targeting, these enzymes can cleave genomic DNA to form a double strand break (DSB) or create a nick which can be repaired by one of two repair pathways, non-homologous end joining (NHEJ) or homologous recombination (HR). The NHEJ pathway can potentially result in specific mutations, deletions, insertions or replacement events. The HR pathway results in replacement of the targeted sequence by a supplied donor sequence. Exemplary FokI and methyltransferase-based systems are described in U.S. Pat. No. 10,220,052, the disclosure of which is incorporated by reference herein in its entirety.

In suitable embodiments, the CRISPR-associated nuclease is a Cas9 nuclease, or can be other Cas nucleases such as Cas12 nuclease, Cas13 nuclease, Cas14 nuclease, etc. In embodiments, the Cas9 nuclease is a Cas9 nuclease that has reduced immunogenicity, such as disclosed in U.S. Published Patent Application No. 2018-0319850, the disclosure of which is incorporated by reference herein in its entirety.

The Clustered Regularly Interspaced Short Palindromic Repeats (CRISPR) and associated proteins (CRISPR-associated nucleases, or Cas proteins), which comprise the CRISPR-Cas system, were first identified in selected bacterial species and form part of a prokaryotic adaptive immune system. See Sorek, et al., “CRISPR—a widespread system that provides acquired resistance against phages in bacteria and archaea,” Nat. Rev. Microbial. 6(3)181-6 (2008), which is incorporated by reference herein in its entirety. CRISPR-Cas systems have been classified into three main types: Type I, Type II, and Type III. The main defining features of the separate Types are the various cas genes, and the respective proteins they encode, that are employed. The cas1 and cas2 genes appear to be universal across the three main Types, whereas cas3, cas9, and cas10 are thought to be specific to the Type I, Type II, and Type III systems, respectively. See, e.g., Barrangou, R. and Marraffini, L. A., “CRISPR-Cas systems: prokaryotes upgrade to adaptive immunity,” Mol. Cell. 54(2):234-44 (2014), which is incorporated by reference herein in its entirety.

In general, the CRISPR-Cas system functions by capturing short regions of invading viral or plasmid DNA and integrating the captured DNA into the host genome to form so-called CRISPR arrays that are interspaced by repeated sequences within the CRISPR locus. This acquisition of DNA into CRISPR arrays is followed by transcription and RNA processing.

Depending on the bacterial species, CRISPR RNA processing proceeds differently. For example, in the Type II system, originally described in the bacterium Streptococcus pyogenes, the transcribed RNA is paired with a transactivating RNA (tracrRNA) before being cleaved by RNase III to form an individual CRISPR-RNA (crRNA). The crRNA is further processed after binding by the Cas9 nuclease to produce the mature crRNA. The crRNA/Cas9 complex subsequently binds to DNA containing sequences complimentary to the captured regions (termed protospacers). The Cas9 protein then cleaves both strands of DNA in a site-specific manner, forming a double-strand break (DSB). This provides a DNA-based memory, resulting in rapid degradation of viral or plasmid DNA upon repeat exposure and/or infection. The native CRISPR system has been comprehensively reviewed (see, e.g., Barrangou, R. and Marraffini, L. A., 2014).

Since its original discovery, multiple groups have done extensive research around potential applications of the CRISPR system in genetic engineering, including gene editing (Jinek et al., “A programmable dual-RNA-guided DNA endonuclease in adaptive bacterial immunity,” Science 337(6096):816-21 (2012); Cong et al., “Multiplex genome engineering using CRISPR/Cas systems,” Science 339(6121):819-23 (2013); and Mali et al., “RNA-guided human genome engineering via Cas9,” Science 339(6121):823-26; each of which is incorporated by reference herein in its entirety). One major development was utilization of a chimeric RNA to target the Cas9 protein, designed around individual units from the CRISPR array fused to the tracrRNA. This creates a single RNA species, called a small guide RNA (gRNA) where modification of the sequence in the protospacer region can target the Cas9 protein site-specifically. Considerable work has been done to understand the nature of the base-pairing interaction between the chimeric RNA and the target site, and its tolerance to mismatches, which is highly relevant in order to predict and assess off-target effects (see, e.g., Fu et al., “Improving CRISPR-Cas nucleases using truncated guide RNAs,” Nature Biotechnology 32(3):279-84 (2014), and supporting material, which is incorporated by reference herein in its entirety).

The CRISPR-Cas9 gene editing system has been used successfully in a wide range of organisms and cell lines, both in order to induce double-strand break formation using the wild type Cas9 protein or to nick a single DNA strand using a mutant protein termed Cas9n/Cas9 D10A (see, e.g., Mali et al., (2013) and Sander and Joung, “CRISPR-Cas systems for editing, regulating and targeting genomes,” Nature Biotechnology 32(4):347-55 (2014), each of which is incorporated by reference herein in its entirety). While double-strand break (DSB) formation results in creation of small insertions and deletions (indels) that can disrupt gene function, the Cas9n/Cas9 D10A nickase avoids indel creation (the result of repair through non-homologous end-joining) while stimulating the endogenous homologous recombination machinery. Thus, the Cas9n/Cas9 D10A nickase can be used to insert regions of DNA into the genome with high-fidelity.

As described herein, suitably the CRISPR-associated nuclease that is inserted into the genome of the T-cell is a Cas9 nuclease. By placing the Cas9 nuclease under the control of a controllable promoter, the nuclease can be kept dormant or silent prior to its desired use as a gene editing tool. As used herein a “controllable promoter” refers to promoter that can be turned on or off, depending on the desired control of the gene that is under control of the promoter.

In addition to a Cas9 nuclease, Cas12, Cas13 and Cas14 nucleases can also be utilized in the methods described herein. Cas12 nuclease creates staggered cuts in dsDNA (5 nucleotide 5′ overhand dsDNA break). Cas12 processes its own guide RNAs, leading to increased multiplexing ability. Cas13t targets RNA, not DNA. Once it is activated by a ssRNA sequence bearing complementarity to its crRNA spacer, it unleashes a nonspecific RNase activity and destroys all nearby RNA regardless of their sequence. See, e.g., Yan et al., “CRISPR-Cas12 and Cas13: the lesser known siblings of CRISPR Cas9,” Cell Biology and Toxicology pages 1-4 (Aug. 29, 2019), the disclosure of which is incorporated by reference herein in its entirety.

In additional embodiments, an inactivated Cas9 enzyme (dCas9) can be linked to an active endonuclease and utilized in the methods described herein, including for example, a dCas9-FokI fusion.

As used herein “under control” refers to a gene being regulated by a “promoter,” “promoter sequence,” or “promoter region,” which refers to a DNA regulatory region/sequence capable of binding RNA polymerase and initiating transcription of a downstream coding or non-coding gene sequence. In other words, the promoter and the gene are in operable combination or operably linked. As referred to herein, the terms “in operable combination”, “in operable order” and “operably linked” refer to the linkage of nucleic acid sequences in such a manner that a promoter capable of directing the transcription of a given gene and/or the synthesis of a desired protein molecule is produced. The term also refers to the linkage of amino acid sequences in such a manner so that a functional protein is produced.

In some examples of the present disclosure, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes.

Various promoters, including inducible promoters, may be used to drive the gene expression, e.g., in the host T-Cell or vectors of the present disclosure. In some embodiments, the promoter is not a leaky promoter, i.e., the promoter is not constitutively expressing any of the gene products as described herein. In other embodiments as described herein, the promoter is a constitutive promoter, which initiates mRNA synthesis independent of the influence of an external regulation. In exemplary embodiments, the promoters used to control the engineered nucleases are inducible promoters. “Inducible promoters” refers to a group of promoters that can enhance the expression of exogenous genes under the stimulation of specific physical, chemical, or pathogen signals. Exemplary inducible promoters that can be used to control the engineered nuclease in embodiments hereof, include, but are not limited to, a 4HT inducible promoter, a rapamycin inducible promoter, a hormone response element, a TET-on system, or a glutamate inducible promoter.

Suitably, the promoters used to control the engineered nucleases are derepressible promoters. As used herein, a “derepressible promoter” refers to a structure that includes a functional promoter and additional elements or sequences capable of binding to a repressor element to cause repression of the functional promoter. “Repression” refers to the decrease or inhibition of the initiation of transcription of a downstream coding or non-coding gene sequence by a promoter. A “repressor element” refers to a protein or polypeptide that is capable of binding to a promoter (or near a promoter) so as to decrease or inhibit the activity of the promoter. A repressor element can interact with a substrate or binding partner of the repressor element, such that the repressor element undergoes a conformation change. This conformation change in the repressor element takes away the ability of the repressor element to decrease or inhibit the promoter, resulting in the “derepression” of the promoter, thereby allowing the promoter to proceed with the initiation of transcription. A “functional promoter” refers to a promoter, that absent the action of the repressor element, would be capable of initiation transcription. Various functional promoters that can be used in the practice of the present invention are known in the art, and include for example, PCMV, PH1, P19, P5, P40 and promoters of Adenovirus helper genes (e.g., E1A, E1B, E2A, E4Orf6, and VA).

Exemplary repressor elements and their corresponding binding partners that can be used as derepressible promoters are known in the art, and include systems such as the cumate gene-switch system (CuO operator, CymR repressor and cumate binding partner) (see, e.g., Mullick et al., “The cumate gene-switch: a system for regulated expression in mammalian cells,” BMC Biotechnology 6:43 (1-18) (2006), the disclosure of which is incorporated by reference herein in its entirety, including the disclosure of the derepressible promoter system described therein) and the TetO/TetR system described herein (see, e.g., Yao et al., “Tetracycline Repressor, tetR, rather than the tetR-Mammalian Cell Transcription Factor Fusion Derivatives, Regulates Inducible Gene Expression in Mammalian Cells,” Human Gene Therapy 9:1939-1950 (1998), the disclosure of which is incorporated by reference herein in its entirety). In exemplary embodiments, the derepressible promoters comprise a functional promoter and either one two tetracycline operator sequences (TetO or TetO2). In such embodiments, the nucleic acid introduced into the T-cells further includes a tetracycline repressor protein to control the TetO derepressible system.

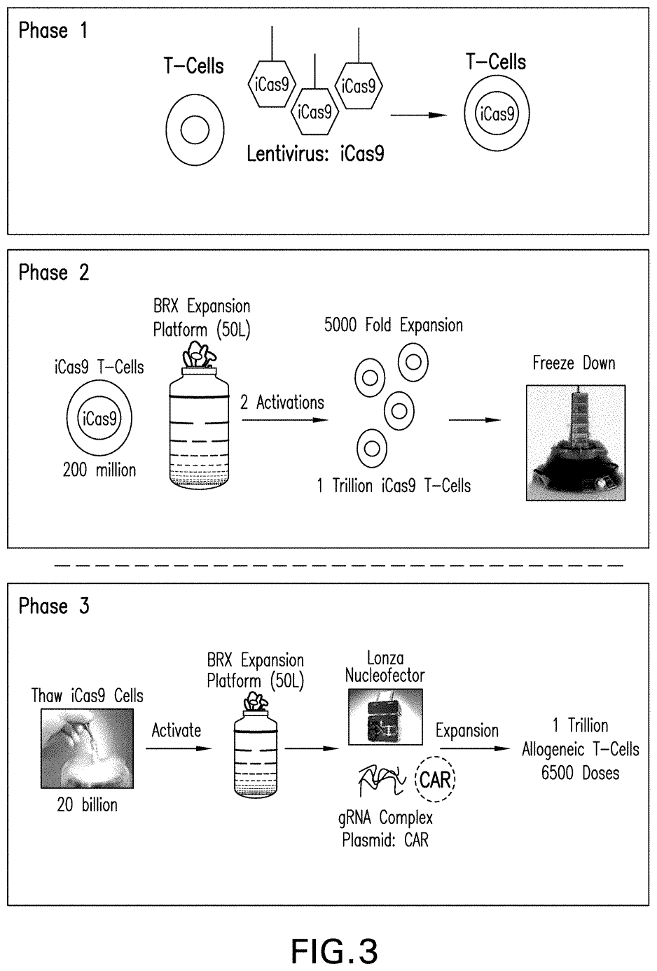

In exemplary embodiments, as shown in FIG. 3, in phase 1, T-cells can be transfected with an inducible Cas9 (iCas9) (or Cas9 under the control of a derepressible promoter) via a viral system, such as a lentivirus. This results in a T-cell that includes a genomically integrated Cas9 (or other engineered nuclease).

In phase 2, as shown in FIG. 3, suitably the iCas9 T-cells (or T-cells containing another engineered nuclease), can be expanded using various methods for cell expansion. Expansion methods of the T-cells described herein can utilize any suitable reactor(s) including but not limited to stirred tank bioreactor, airlift, fiber, microfiber, hollow fiber, ceramic matrix, fluidized bed, fixed bed, and/or spouted bed bioreactors. As used herein, “reactor” can include a fermenter or fermentation unit, or any other reaction vessel and the term “reactor” is used interchangeably with “fermenter.” The term fermenter or fermentation refers to both microbial and mammalian cultures. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments, the expansion suitably includes activation of the T-cells. As described herein, as the T-cell receptor has not been removed from the engineered T-cell line, the cells can still be activated. In vivo, antigen-presenting cells (APCs), such as dendritic cells, act as the stimulus for T-Cell activation through the interaction of the T-Cell Receptor (TCR) with the APC major histocompatibility complex (MHC). TCR associates with CD3, a T-Cell co-receptor that helps to activate both cytotoxic T-Cells (e.g., CD8+ naïve T-Cells) and T helper cells (e.g., CD4+ naïve T-Cells). In general, T-Cell activation follows a two-signal model, requiring stimulation of the TCR/CD3 complex as well as a co-stimulatory receptor.

Non-limiting examples of co-stimulatory molecules for T-Cells include CD28, which is a receptor for CD80 and CD86 on the membrane of APC; and CD278 or ICOS (Inducible T-cell COStimulator), which is a CD28 superfamily molecule expressed on activated T-Cells that interacts with ICOS-L. Thus, in some embodiments, the co-stimulatory molecule is CD28. In other embodiments, the co-stimulatory molecule is ICOS. In vivo, the co-stimulatory signal can be provided by the B7 molecules on the APC, which bind to the CD28 receptor on T-Cells. B7 is a peripheral transmembrane protein found on activated APCs that can interact with CD28 or CD152 surface proteins on a T-Cell to produce a co-stimulatory signal. Thus, in some embodiments, the co-stimulatory molecule is B7.

Various methods of activation are utilized in vitro to simulate T-Cell activation. In embodiments, a T-Cell culture is activated with an activation reagent. In further embodiments, the activation reagent is an Antigen-Presenting Cell (APC). In still further embodiments, the activation reagent is a dendritic cell. Dendritic cells are APCs that process antigen and present it on the cell surface to T-Cells. In some embodiments, the activation reagent is co-cultured with the T-Cell culture. Co-culturing may require separate purification and culturing of a second cell type, which may increase labor requirements and sources of variability. Thus, in some embodiments, alternative activation methods are used.

In some embodiments, the activation reagent is an antibody. In some embodiments, the cell culture is activated with an antibody bound to a surface, including a polymer surface, including a bead. In further embodiments, the one or more antibodies is an anti-CD3 and/or anti-CD28 antibody. For example, the beads may be magnetic beads such as, e.g., DYNABEADS, coated with anti-CD3 and anti-CD28. The anti-CD3 and anti-CD28 beads can suitably provide the stimulatory signals to support T-Cell activation. See, e.g., Riddell 1990; Trickett 2003.

In other embodiments, the cell culture is activated with a soluble antibody. In further embodiments, the soluble antibody is a soluble anti-CD3 antibody. OKT3 is a murine monoclonal antibody of the immunoglobulin IgG2a isotype and targets CD3. Thus, in some embodiments, the soluble anti-CD3 antibody is OKT3. OKT3 is further described in, e.g., Dudley 2003; Manger 1985; Ceuppens 1985; Van Wauwe 1980; Norman 1995.

In some embodiments, the co-stimulatory signal for T-Cell activation is provided by accessory cells. Accessory cells may include, for example, a Fc receptor, which enables cross-linking of the CD3 antibody with the TCR/CD3 complex on the T-Cell. In some embodiments, the cell culture is a mixed population of peripheral blood mononuclear cells (PBMCs). PBMC may include accessory cells capable of supporting T-Cell activation. For example, CD28 co-stimulatory signals can be provided by the B7 molecules present on monocytes in the PBMC. Accordingly, in some embodiments, the accessory cells include a monocyte or a monocyte-derived cell (e.g., a dendritic cell). In additional embodiments, the accessory cells include B7, CD28, and/or ICOS. Accessory cells are further described in, e.g., Wolf 1994; Chai 1997; Verwilghen 1991; Schwartz 1990; Ju 2003; Baroja 1989; Austyn 1987; Tax 1983.

As described herein, activation reagent may determine the phenotype of the CAR T-Cells produced, allowing for the promotion of a desired phenotype. In some embodiments, the activation reagent determines the ratio of T-Cell subsets, i.e., CD4+ helper T-Cells and CD8+ cytotoxic T-Cells. The cytotoxic CD8+ T-Cells are typically responsible for killing cancer cells (i.e., the anti-tumor response), cells that are infected (e.g., with viruses), or cells that are damaged in other ways. CD4+ T-Cells typically produce cytokines and help to modulate the immune response, and in some cases may support T-Cell lysis. CD4+ cells activate APCs, which then primes naïve CD8+ T-Cells for the anti-tumor response. Accordingly, in embodiments, the methods of the present disclosure further include producing CAR T-Cells of a pre-defined phenotype (i.e., promoting cells of a desired phenotype). The pre-defined phenotype may be, for example, a pre-defined ratio of CD8+ cells to CD4+ cells. In some embodiments, the ratio of CD8+ cells to CD4+ cells in a population of CAR T-Cells is about 1:1, about 0.25:1, or about 0.5:1. In other embodiments, the ratio of CD8+ cells to CD4+ cells in a population of CAR T-Cells is about 2:1, about 3:1, about 4:1, or about 5:1.

In some embodiments, the T cell culture is expanded to a pre-defined culture size (i.e., number of cells). The pre-defined culture size may include a sufficient number of cells suitable for clinical use, i.e., transfusion into a patient, research and development work, etc. In some embodiments, a clinical or therapeutic dose of T-cells for administration to a patient is about 10⁵ cells, about 10⁶ cells, about 10⁷ cells, about 10⁸ cells, about 10⁹ cells, or about 10¹⁰ cells. In some embodiments, the method produces at least 1, at least 2, at least 3, at least 4, at least 5, at least 10, at least 15, at least 20, at least 25, at least 30, at least 35, at least 40, at least 45, at least 50, at least 60, at least 70, at least 80, at least 90, or at least 100 clinical doses of T-cells (and thus ultimately CAR T-cells). In embodiments, the number of T-cells produced by the methods described herein is at least about 100 million (i.e., 100*10⁶) cells, or at least about 1 billion (i.e., 1*10⁹) cells, at least about 50 billion, at least about 100 billion, at least about 250 billion, at least about 500 billion, at least about 750 billion, or at least about 1 trillion (i.e., 1*10¹²) cells, including at least about 2 trillion, at least about 3 trillion, at least about 4 trillion, at least about 5 trillion, or at least about 10 trillion T-cells.

Following the expansion of the T-Cells, suitably the cells are prepared for storage, including for example, freezing the expanded T-cell line following the expanding. Methods of freezing the expanded cells are known in the art, and include the use of liquid nitrogen, dry ice, and can include various lyophilization procedures. Suitably the cells are frozen at a temperature of about −80° C. to about 0° C., and can include the use of a cryoprotective agent such as dimethylsulfoxide (DMSO). Cells can be stored in a frozen state for weeks, months and even years, until a desired time after which they can be thawed for further processing and/or genetic modification as described herein.

In further embodiments, provided herein is a method of producing a genetically modified T-cell line, comprising: introducing a nucleic acid molecule encoding a CRISPR-associated nuclease under the control of a controllable promoter into a T-cell line; integrating the nucleic acid molecule into the genome of the T-cell line; and expanding the T-cell line. As described herein, the nucleic acid molecule encoding a CRISPR-associated nuclease is RNA that is reverse transcribed to DNA, and then integrated into the genome of the cell. As described herein, suitably the CRISPR-associated nuclease is a Cas9 nuclease or a Cas12 nuclease.

As described herein, following the expansion, the T-cell can be frozen if desired and stored. The stored cells are then suitably thawed prior to further processing.

As described herein, the methods can further include inducing expression of the CRISPR-associated nuclease by activating the controllable promoter. In the case of an inducible promoter, such as a 4HT inducible promoter, a rapamycin inducible promoter, a hormone response element, or a glutamate inducible promoter, the promoter is induced by the addition of, for example, 4-hydroxytamoxifen, rapamycin, a hormone, or glutamate, respectively. In the case of a derepressible promoter, such as the TetO sequence described herein coupled to a CMV promoter, the addition of doxycycline removes the repression, and allows the gene (engineered nuclease) to be expressed via the CMV promoter. Suitably, the nucleic acid molecule that encodes the Cas9 also encodes a TetR repressor element, suitably under the control of another promoter system, such as a constitutive promoter like the hPGK promoter. The controllable promoter can also be a Tet-on system, including the use of a TRE3G promoter sequence, as described herein.

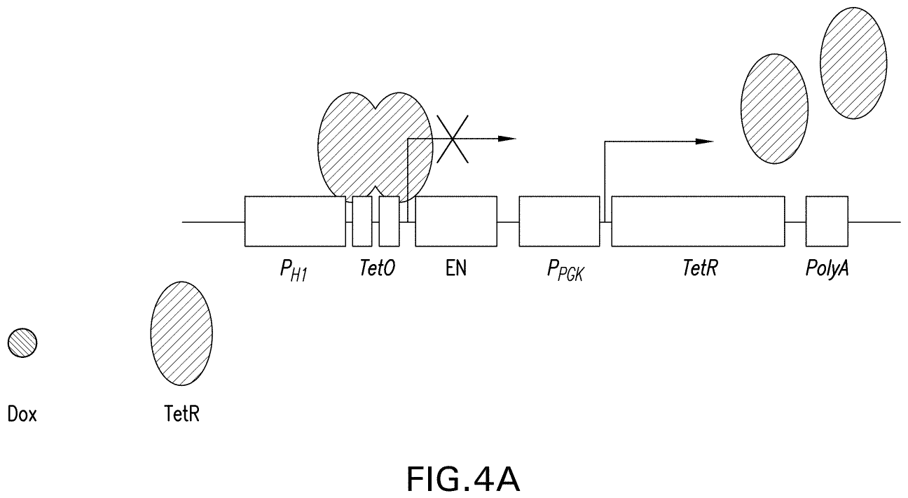

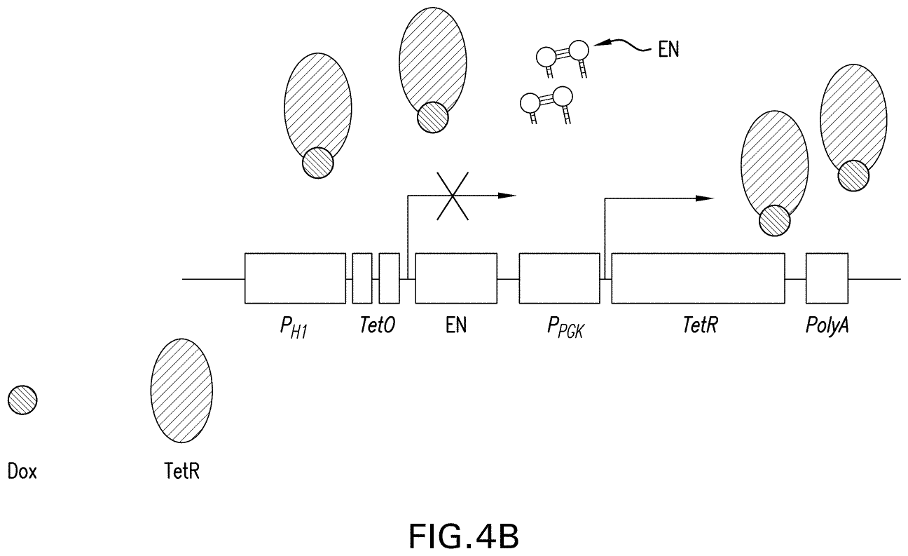

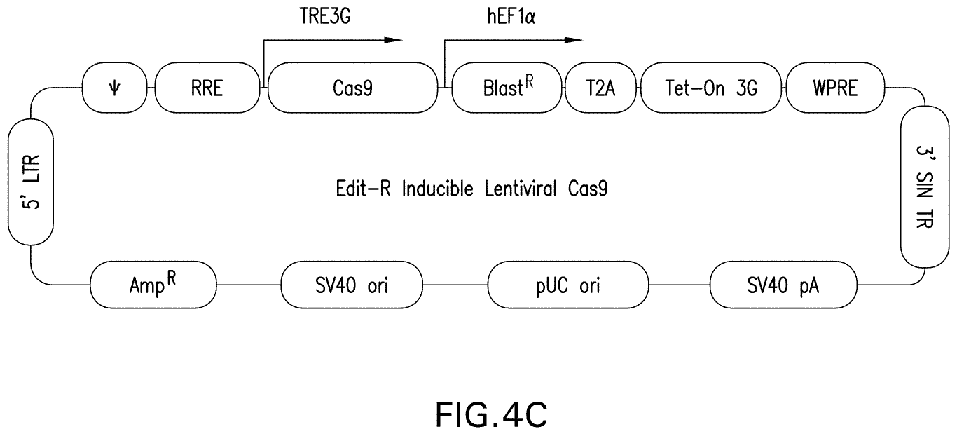

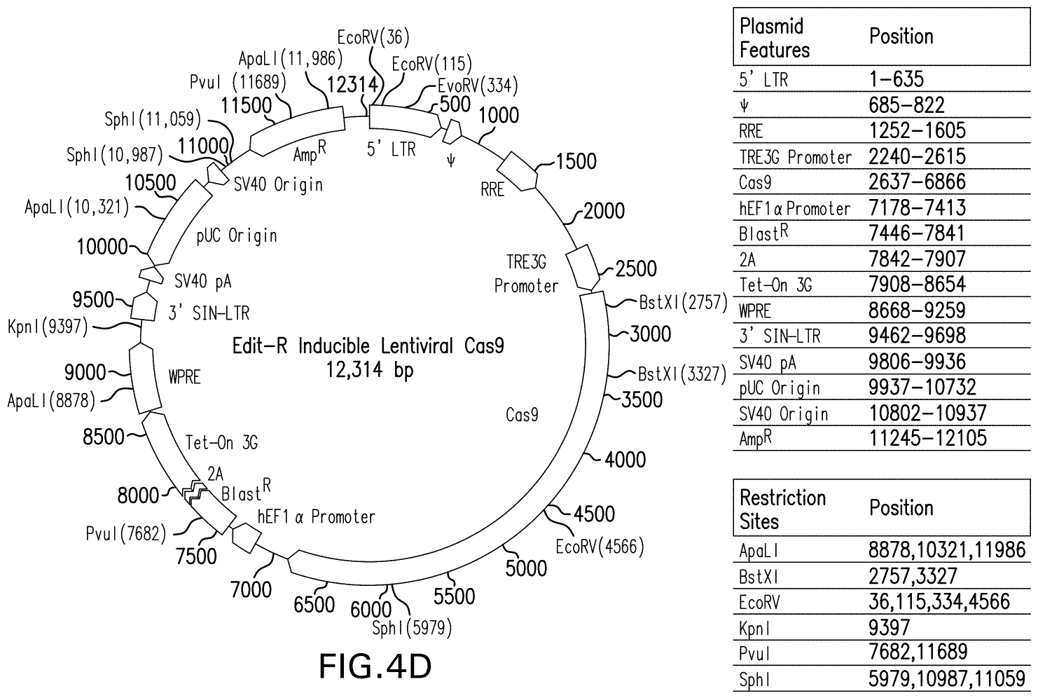

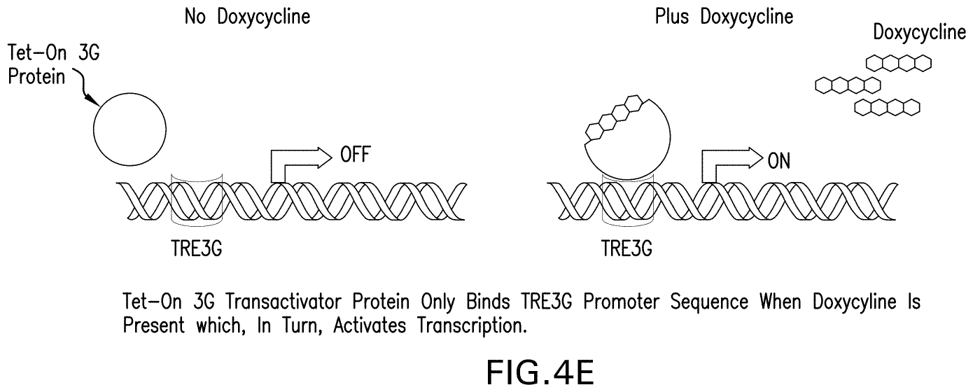

FIGS. 4A-4B illustrates an exemplary derepressible system, the TetO system described herein. As shown in FIG. 4A, two TetO sequences (along with a promoter sequence) are suitably oriented prior to the engineered nuclease (EN). Upon binding of a tetracycline repressor protein (TetR—the repressor elements for the TetO sequences), to the TetO sequences, the promoters (e.g., CMV) are repressed. That is, little or no transcription takes place from these promoters. As shown in FIG. 4B, upon binding of a binding partner for TetR (suitably Doxycycline (Dox)), the TetR proteins change conformation, release from the TetO sequences, and the functional promoters (e.g., CMV) begin its normal transcription process, as they would naturally, resulting in production of the engineered nuclease (EN).

FIG. 4C shows an exemplary inducible vector (e.g., a lentiviral vector) that can be used to integrate the inducible engineered nuclease, in this case a Cas9 nuclease under the control of a TET-on operating system (TRE3G), allowing for expression of the Cas9 in the cell upon induction with Doxycycline. FIG. 4D shows a more detailed vector map.

FIG. 4E shows the operation of the TET-on operating systems, TRE3G. As shown, the Tet-On 3G transactivator protein, in the absence of doxycycline, does not bind to the TRE3G promoter sequence. In the presence of doxycycline, the Tet-On 3G transactivator protein is able to bind to the TRE3G promoter sequence, which activates transcription, and causes the expression of Cas9 nuclease (or other nuclease if desired).

As noted in FIG. 4D, the controllable systems described herein for introducing a nuclease (e.g., Cas9 or Cas12) also suitably include a selectable marker, such as an antibiotic resistance gene (e.g., Ampicillin resistance) to allow for production of inducible-nuclease containing cells (including T-cells) that can readily be selected and enriched. The other controllable systems described herein (other inducible systems as well as derepressible systems) can also be used in combination with selectable markers to allow for selection of nuclease expressing cells, and then enrichment to provide a pure cell population with the desired nuclease (e.g., Cas9 or Cas12) integrated into the genome.

As shown in FIG. 3, phase 3, in addition to activating the expression of the Cas9 nuclease, a guide-RNA and a gene of interest are also introduced into the expanded T-cell line. As described herein, this introduction of the guide-RNA and gene of interest can be introduced via a transfection mechanism such as nucleofection.

As a result of this introduction of a guide-RNA and the gene of interest, the T-cell receptors are suitably knocked out, and the gene of interest is introduced into the genome of the T-cell line. After introduction of the gene of interest, the T-cells are suitably expanded, then the genetically modified T-cell line is recovered. Methods for expansion are known in the art and described here. Methods of recovering the desired cells include various filtration methods, centrifugation, as well as cell isolation and washing.

Knocking out the T-cell receptors suitably includes knocking out the TRAC gene (T-cell receptor alpha subunit), which leads to the ablation of the entire T-cell receptor. As described herein, various guide-RNA sequences can be used to knock out the TRAC gene, and includes those noted in the Examples, as well as other sequences that are readily determined by those of ordinary skill in the art.

As described herein, in order to produce a chimeric antigen receptor T-cell, the gene of interest suitably encodes a chimeric antigen receptor (CAR). As shown in FIG. 2, the genetic editing by the Cas9 nuclease (or Cas12 nuclease) results in the knocking out of the T-Cell receptor, and the expression of the desired CAR on the T-cell. Such T-cells can now be administered to the desired patient population, based on the desired CAR.

Integration of the desired CAR construct into the T-cells suitably occurs at the location of the knock-out of the TRAC gene, such that the CAR is under the control of the endogenous promoter of the TRAC gene.

As described herein, the ability to expand the T-cells to significant numbers prior to inducing the expression of the Cas9, and then subsequent integration of the gene of interest, allows for production of high number of T-cells, on the order of 10⁹-10¹² T cells.

In further embodiments, rather than introducing a gene of interest, one or more genes can be knocked out in the T-cells to create the desired modifications. For example, genes such as programmed cell death protein 1 (PD1) and/or B2M gene (132 microglobulin) responsible for encoding a serum protein found in association with the major histocompatibility complex (MEW) class I heavy chain. Knock-out of such genes can occur using various methods such as antisense, siRNA, microRNA, and other approaches known in the art.

In additional embodiments, provided herein is a method of producing a chimeric antigen receptor (CAR) T-cell line, comprising: introducing a nucleic acid molecule encoding a Cas9 nuclease under the control of a controllable promoter into a T-cell line; integrating the nucleic acid molecule into the genome of the T-cell line; expanding the T-cell line; inducing expression of the Cas9 nuclease by activating the controllable promoter; introducing a guide-RNA and a nucleic acid encoding a chimeric antigen receptor (CAR) into the expanded T-cell line; knocking out expression of a T-cell receptor and introducing the nucleic acid encoding the CAR into the genome of the T-cell line; and recovering the CAR T-cell line.

Examples of various controllable promoters, including inducible promoters and derepressible promotors are described herein, as are methods of inducing expression of the Cas9 nuclease via the introduction of a molecule that induces expression, or that derepresses a derepressible promoter.

In additional embodiments, provided herein is an allogeneic T-cell line, comprising a CRISPR-associated (Cas) nuclease under the control of a controllable promoter integrated into the genome of the T-cell line. As described herein, the Cas nuclease is suitably a Cas9 nuclease.

As described herein, suitably the allogenic T-cell line includes a controllable promoter that is an inducible promoter, including for example a 4HT inducible promoter, a rapamycin inducible promoter, a hormone response element, or a glutamate inducible promoter. The controllable promoter can also be a Tet-on system.

In further embodiments, the controllable promoter can be a derepressible promoter, such as the use of one or more tetracycline operator sequences (TetO). In such embodiments, the T-cell further includes a nucleic acid molecule encoding a tetracycline repressor protein.

As described, the allogenic T-cells prepared in accordance with the embodiments described allow for the production of at least about 10⁹ T-cells, at least about 10¹⁰ T-cells, at least about 10¹¹ T-cells, or in embodiments at least about 10¹² T-cells.

Also provided herein are methods of treating a mammalian subject, suitably a human subject, comprising administering a CAR T-cell prepared using the allogeneic T-cells described herein, as well as CAR T-cells prepared using the methods described herein. Administration to a human subject can include, for example, inhalation, injection, or intravenous administration, as well as other administration methods known in the art.

### EXAMPLES

### Example 1: Transduction of T-Cells with Cas9 Inducible Vector

Three treatment protocols were investigated for use in transducing T-cells with an inducible Cas9 vector. The vector includes a Green Fluorescent Protein (GFP) tag to determine the levels of transduction.

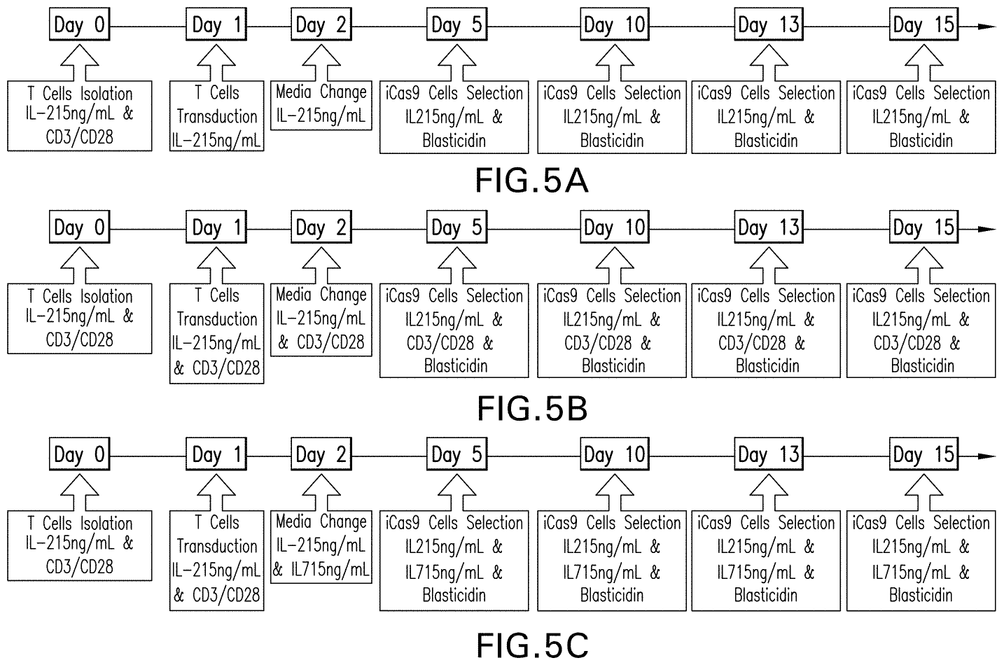

As illustrated in FIGS. 5A-5C, Treatment 1 (IL2) included 24 hr activation with IL-2 at 15 ng/mL & CD3/CD28 on the day of T cells isolation. Following viral transduction, expansion only included IL-2 at 15 ng/mL (IL2). Treatment 2 (IL2+CD3/CD28) included 24 hr activation with IL-2 at 15 ng/mL & CD3/CD28 on the day of T cells isolation. Following transduction, expansion included treatment with IL-2 at 15 ng/mL & CD3/CD28 at every media change. Treatment 3 (IL2+IL) included 24 hr Activation with IL-2 at 15 ng/mL & CD3/CD28 on the day of T cells isolation. Following transduction, cells were treated with IL-2 at 15 ng/mL & CD3/CD28. Expansion was conducted in the presence of only IL-2 & IL-7.

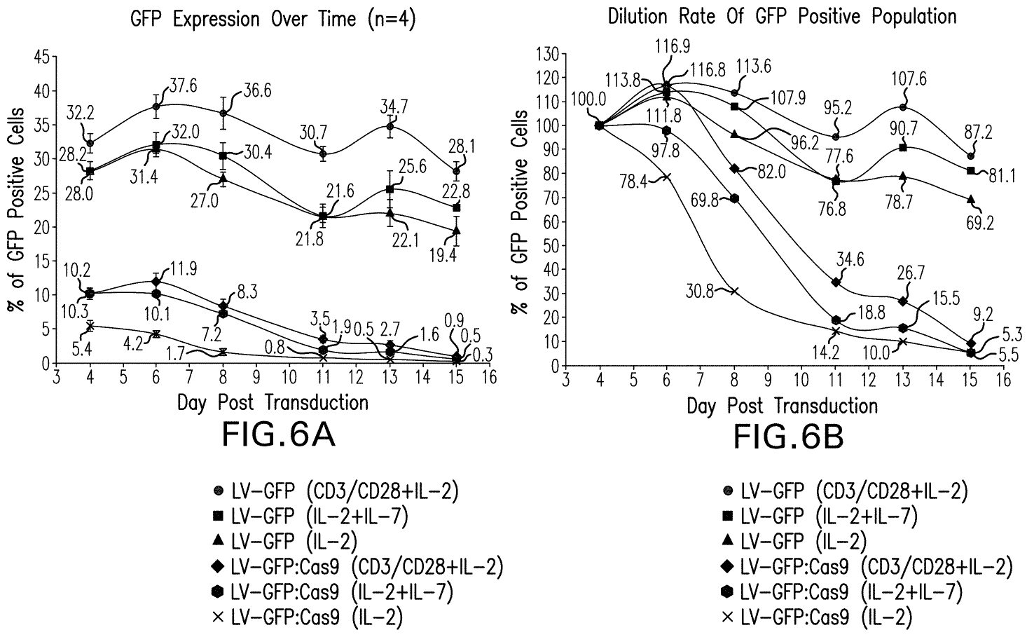

Results of the transduction are provided in FIGS. 6A-6B. As shown in FIG. 6A, the percent of GFP positive cells using the treatment combination of CD3/CD28+IL-2 had the greatest transduction efficiency, with both the control vector (#) and vector that contained the Cas9 nuclease gene (@). Treatment with IL-2+IL-7 also showed a good transduction ($). FIG. 6B shows the dilution rate of the GFP positive population.

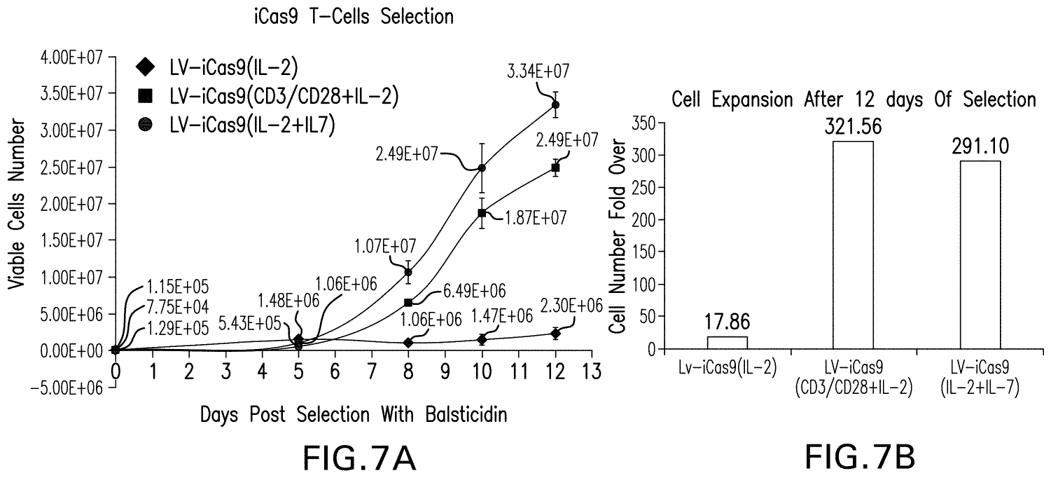

A Balsticidin resistance gene in the Cas9 inducible vector was used to select for cells that include the Cas9 vector correctly inserted into the genome. FIG. 7A shows the number of viable cells for the three treatments described in this Example. As illustrated, treatment with IL-2+IL-7 showed the most viable cells. Upon expansion for 12 days after selection, cells treated with CD3/CD28+IL-2 and IL-2+IL-7 both showed a high number of viable cells. (FIG. 7B).

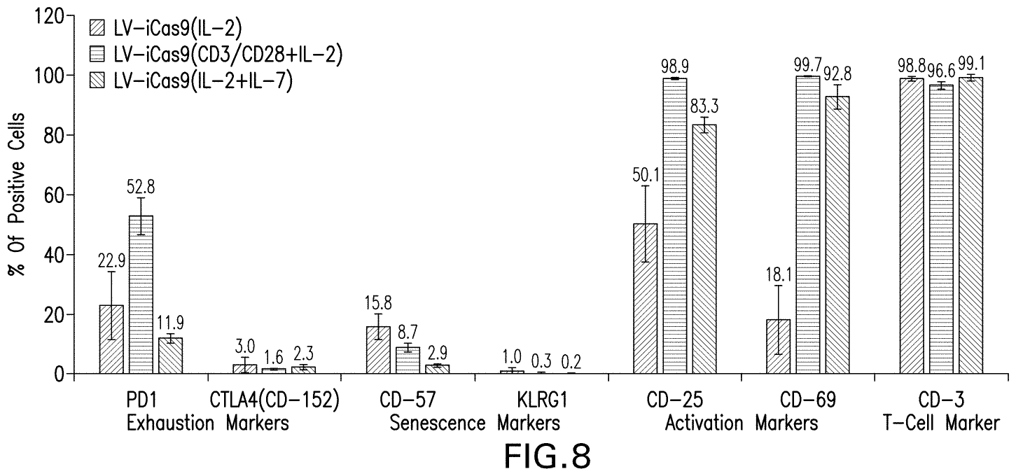

Exhaustion, senescence and activation markers were measured for the three treatment protocols and results are shown in FIG. 8.

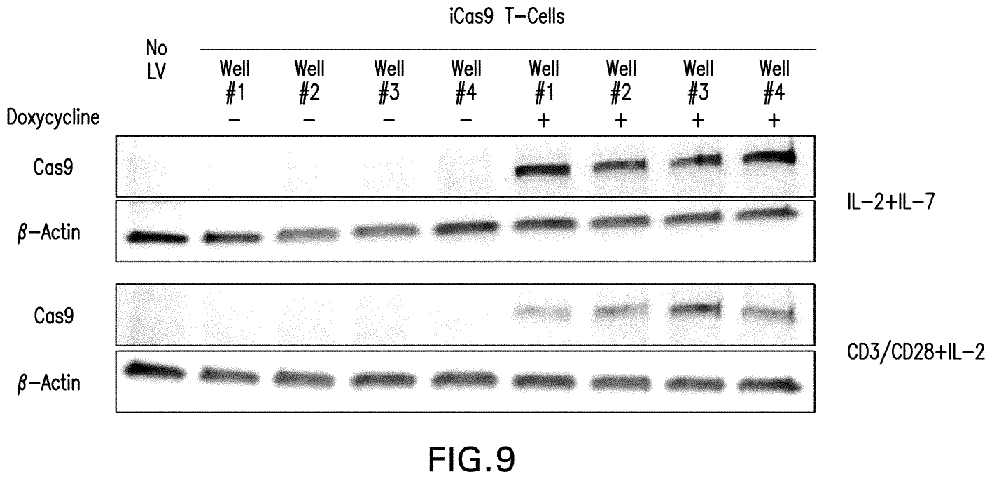

Induction of Cas9 expression was carried out on day 11, post-selection with Blasticidin. Cells were induced for 24 hours with 1 μg/mL of Doxycycline, and the protein lysate was analyzed by Western Blot. As shown in FIG. 9, cell growth with treatment 3 (IL-2+IL-7) showed higher expression of Cas9 upon induction, but cells treatment with CD3/CD28+IL-2 also showed expression of the Cas9 nuclease.

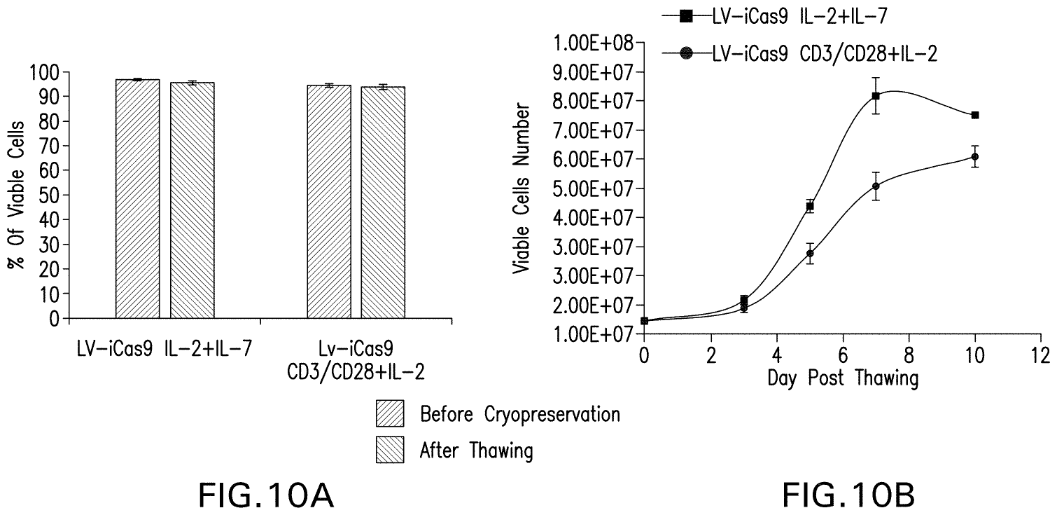

Cells were then frozen, and thawed to determine if insertion of the Cas9 vector had any impact on cell viability. As shown in FIG. 10A, viability was nearly identical before and after cryopreservation for both treatments noted. FIG. 10B shows cell viability days after thawing, indicating that for both treatment, cells were able to proliferate successfully.

### Example 2: Knock-Out of TRAC Gene

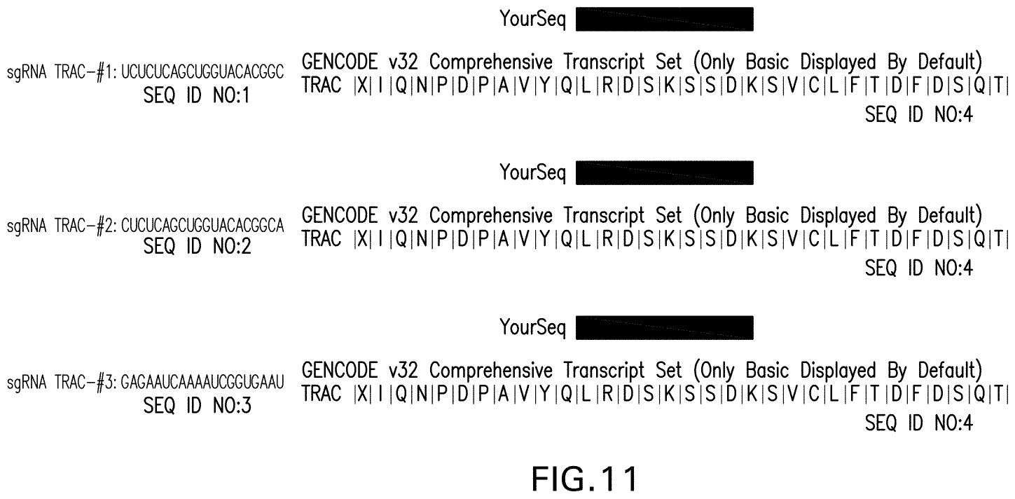

Three sgRNA sequences were selected to investigate the ability to knock-out the TRAC gene in T-cells that had been transduced with the Cas9 vector. FIG. 11 shows the three sgRNA sequences (SEQ ID NOS: 1-3) that were investigated, along with the regions they target in the translated TRAC gene (SEQ ID NO:4). sgRNA sequences were transfected into the T-cell using a nucleofection procedure. Briefly, 1×10⁶ cells in 20 μL were transduced at room temperature with about 3.3 μg of sgRNA using a AMAXA P2 primary cell 4D nucleofector X kit and a EO-115 program.

Knock-out experiments were carried out with sgRNAs TRAC #1-3 (SEQ ID NOs:1-3) and calibrated relative to CD-3. Cas9 T-cells (treated with CD3/CD28 & IL-2, IL-7 at 15 ng/mL, and then selected with Blasticidin at 15 μg/mL) were thawed on Day 1. On Day 2, Cas9 was induced with Doxycyclin (2 μg/mL for 24 hours). On Day 3, the cells with transduced with sgRNA TRAC #1 (SEQ ID NO:1), TRAC #2 (SEQ ID NO:2) and TRAC #3 (SEQ ID NO:3) via nucleofection, and then expanded. On Days 4, 7, and 14, FACS analysis of CD-3 and TCRc43 expression levels were conducted.

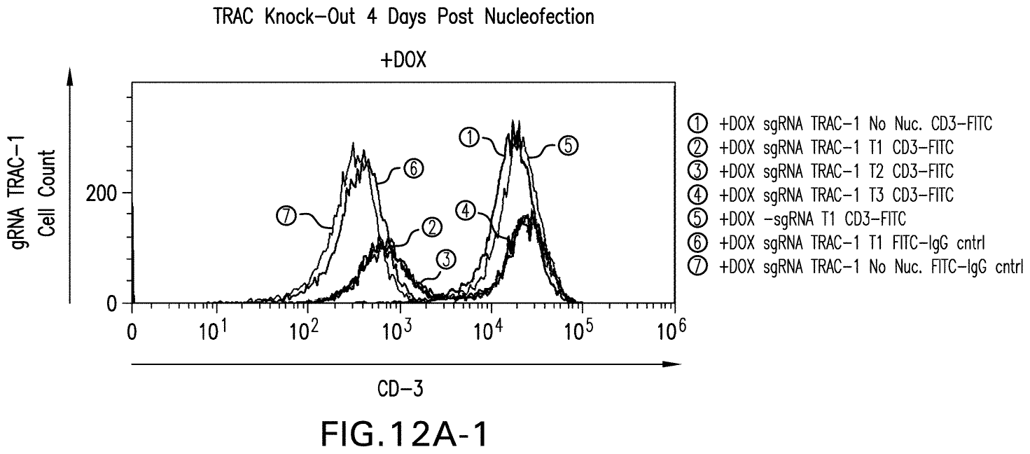

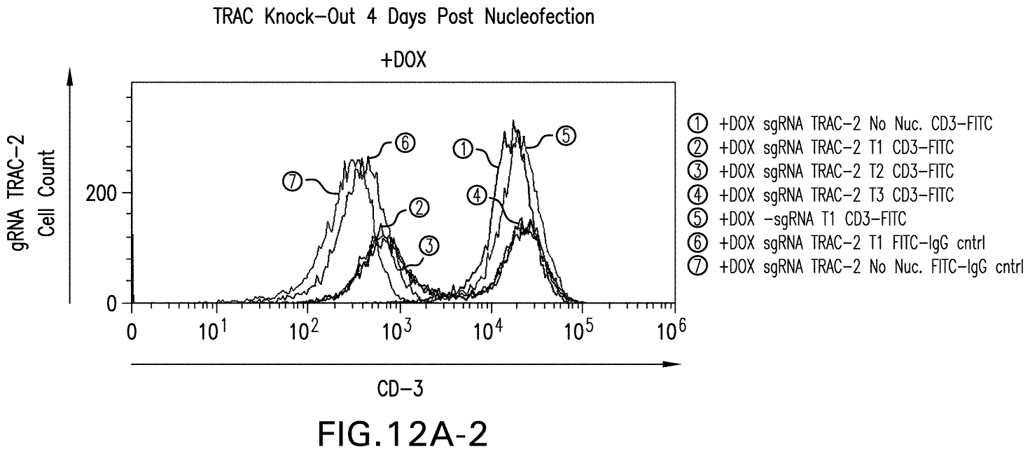

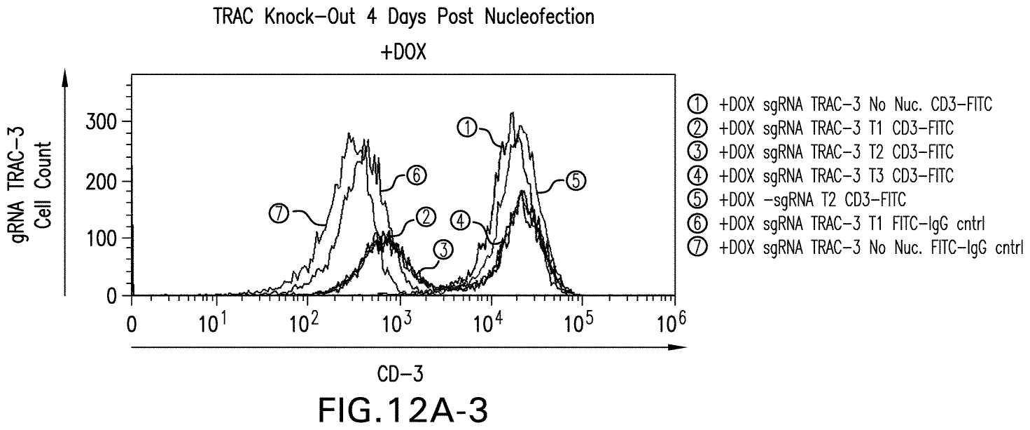

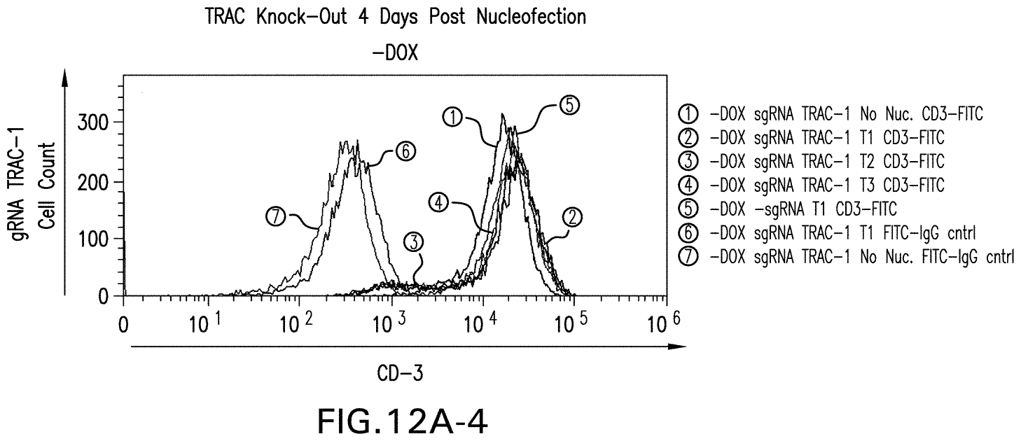

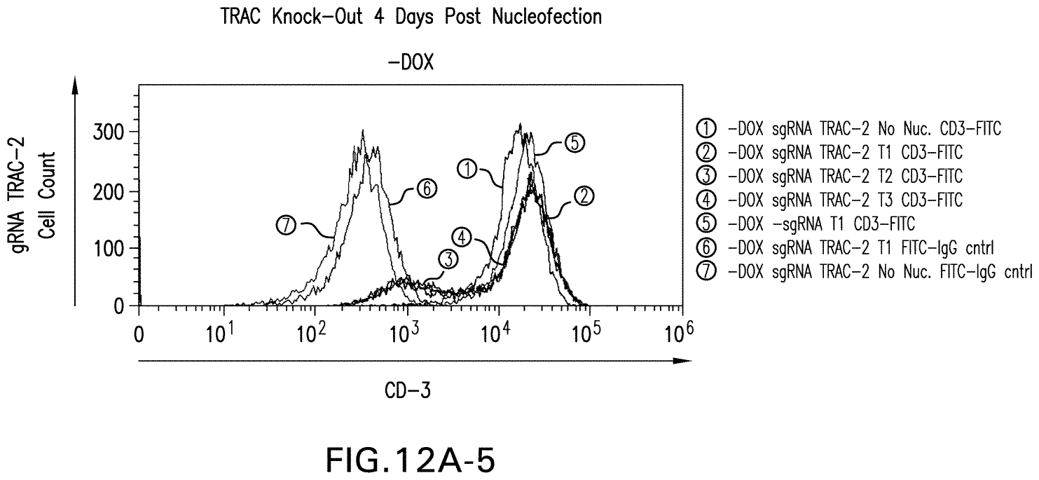

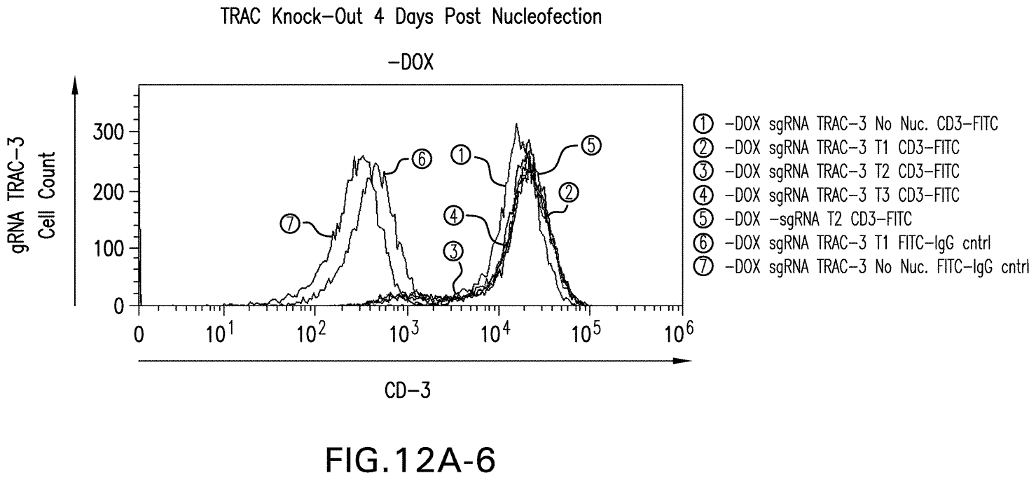

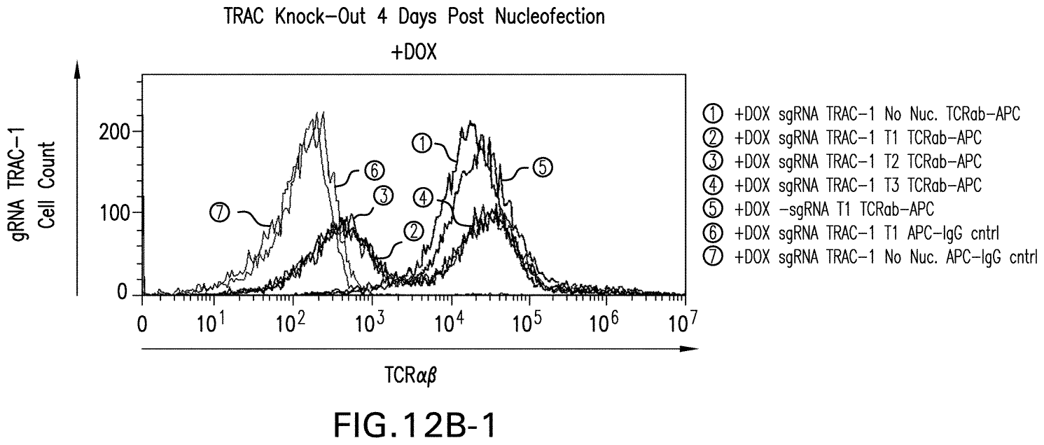

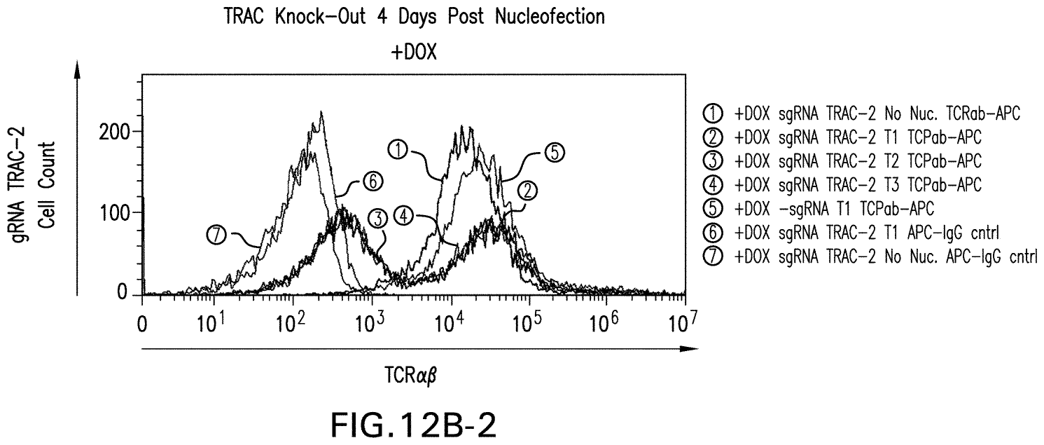

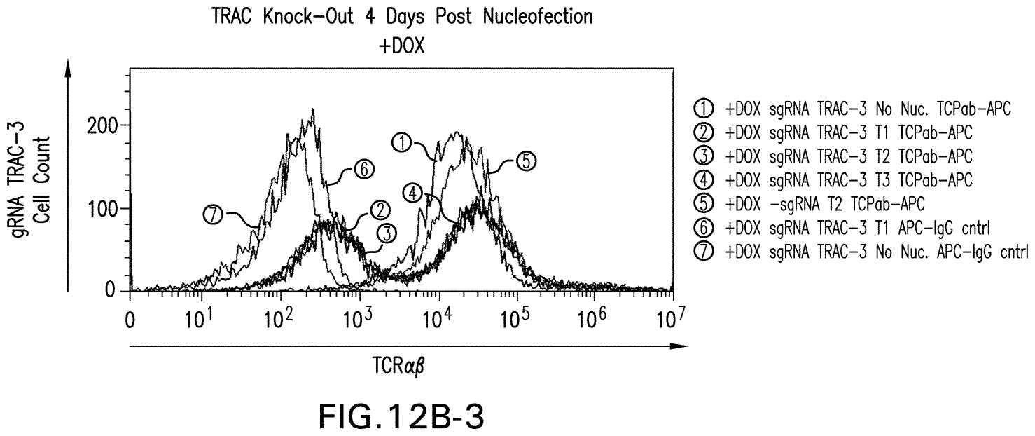

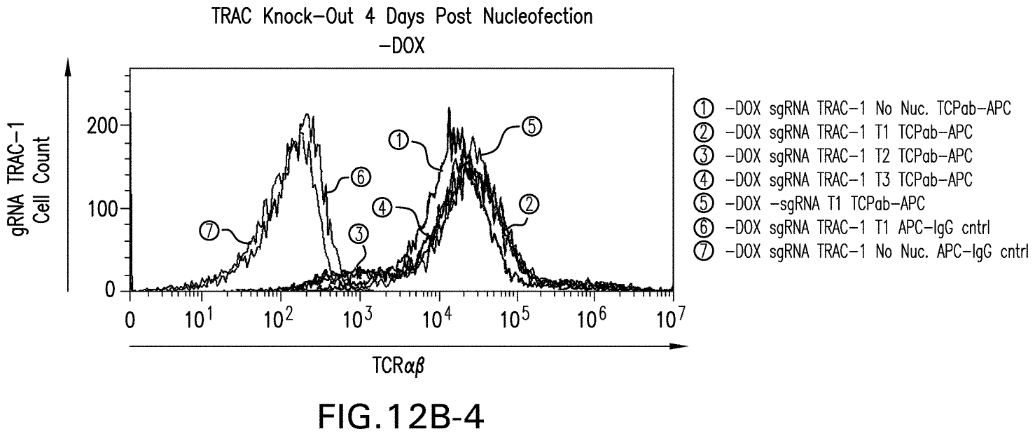

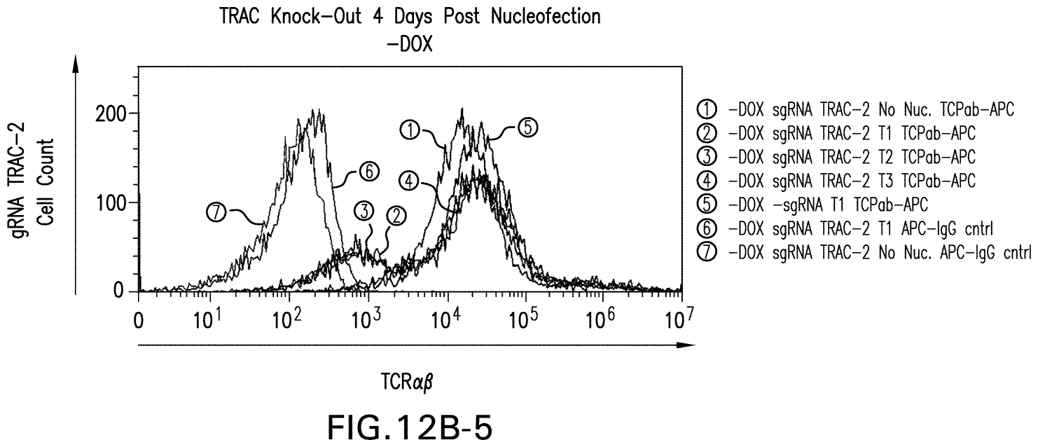

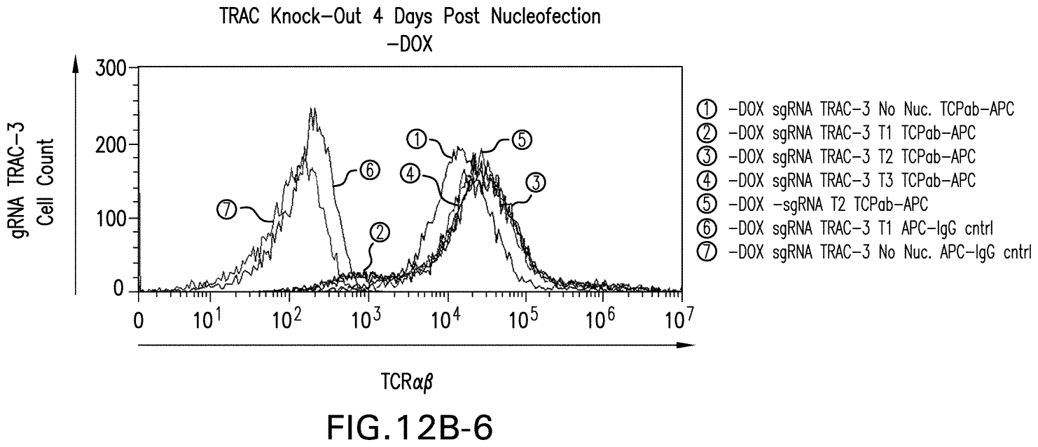

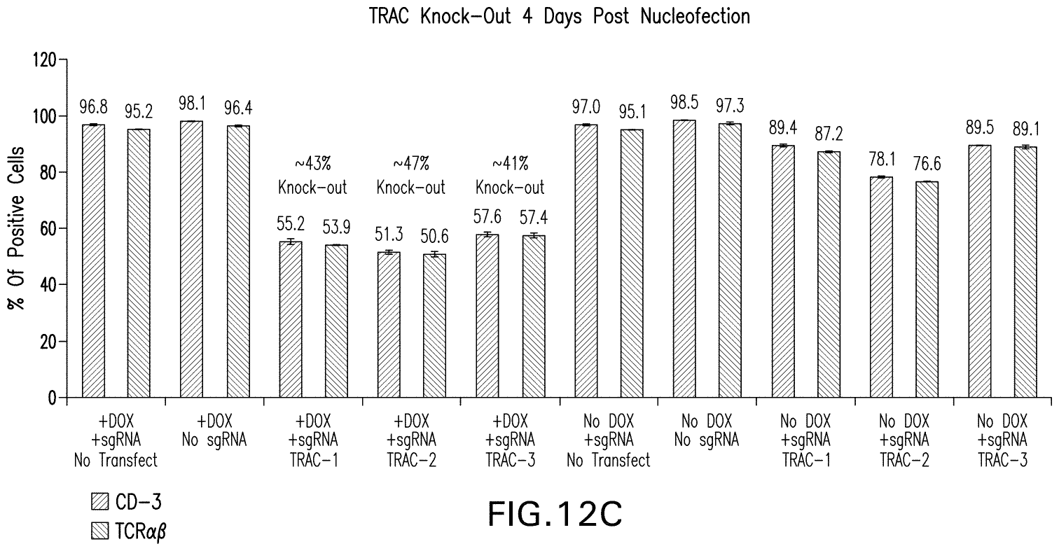

FIGS. 12A-1 through 12B-6 show TRAC knock-out 4 days post nucleofection, with TRAC #1-#3 sgRNA sequences. FIG. 12C shows a compilation of the results. As indicated, each of TRAC #1-TRAC #3 resulted in about 41-47% knock out of the TRAC gene, with TRAC #2 sgRNA showing the highest amount of knock-out (47%).

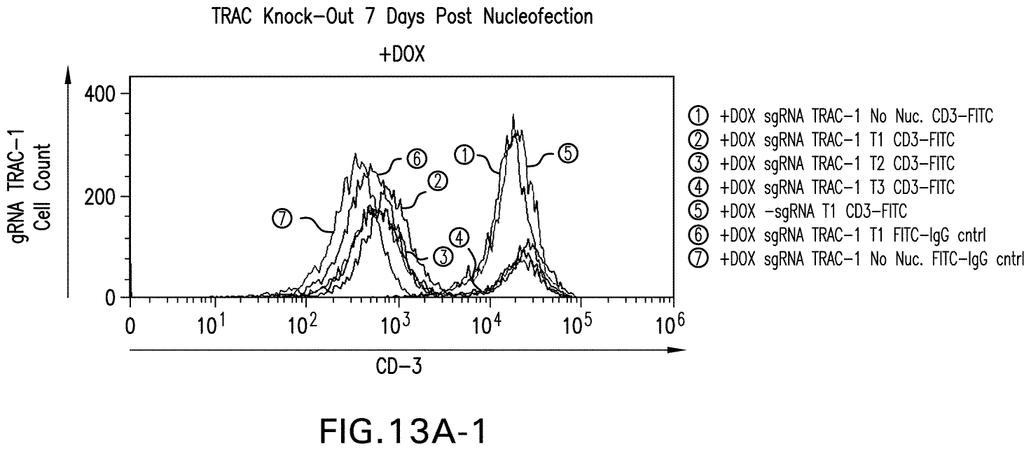

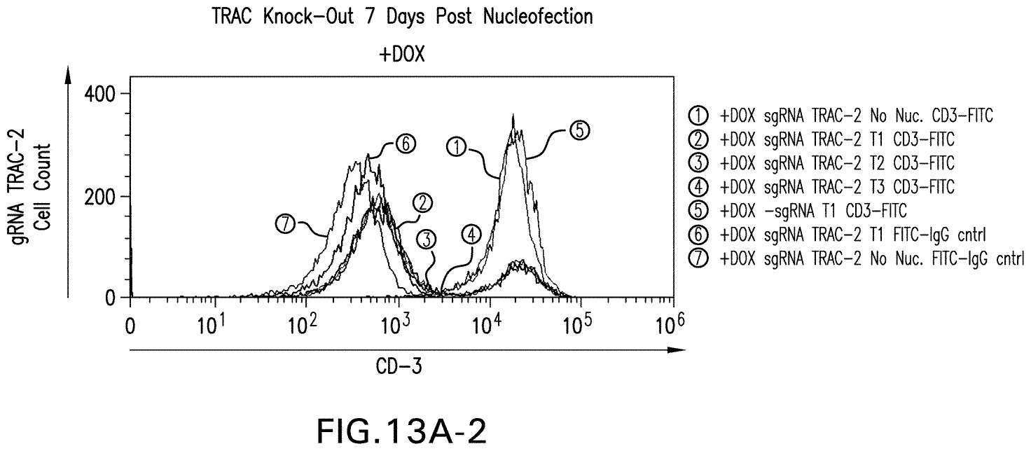

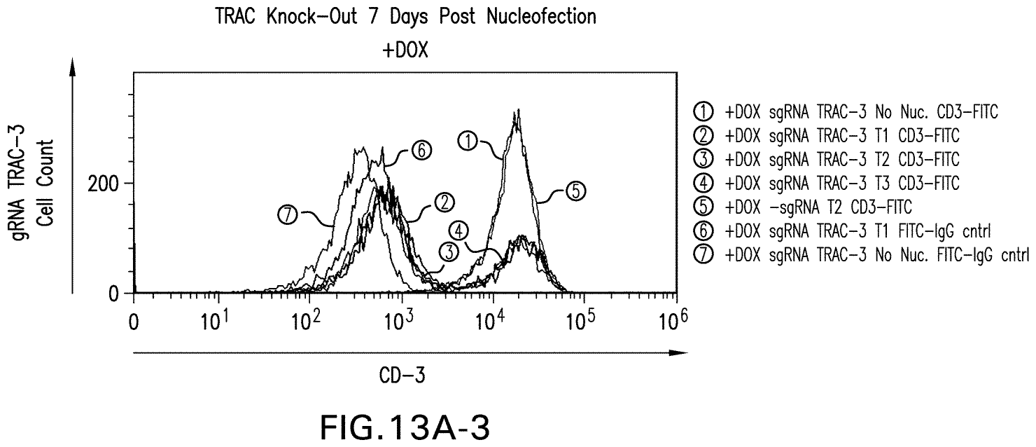

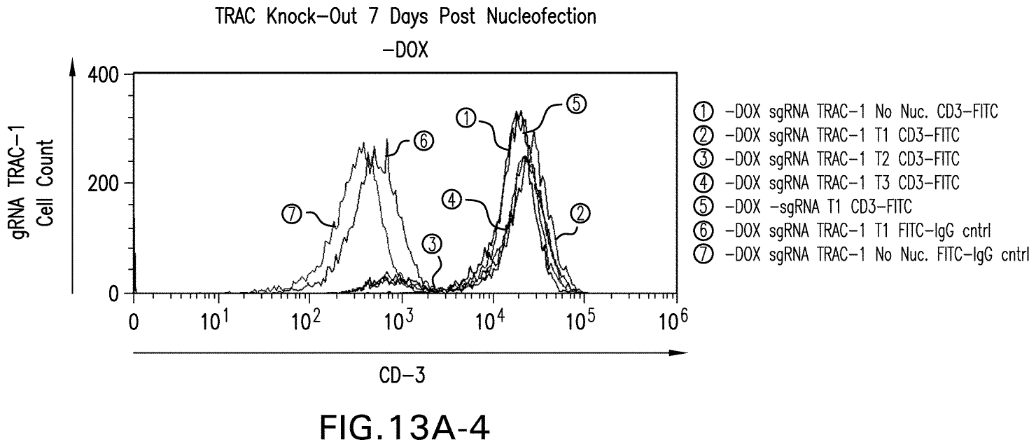

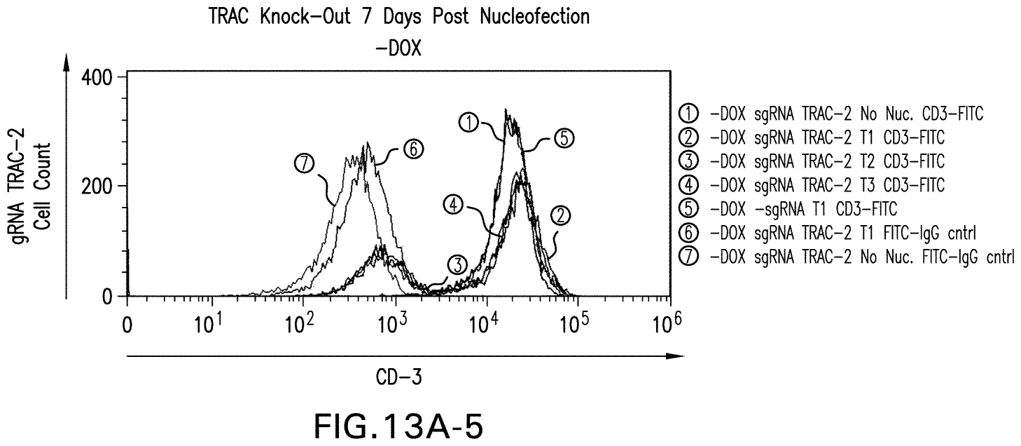

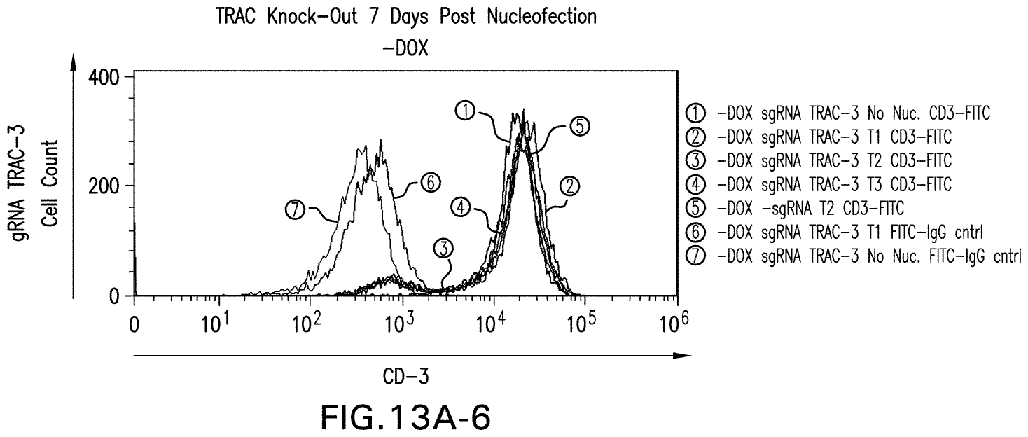

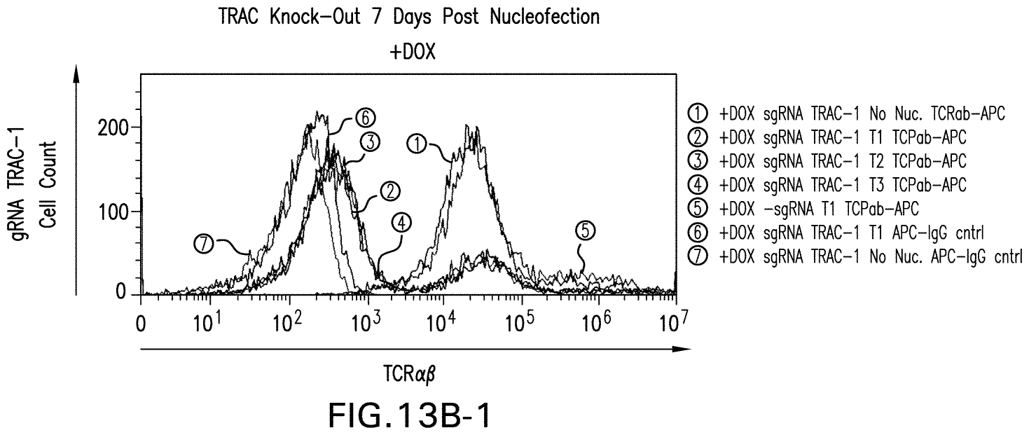

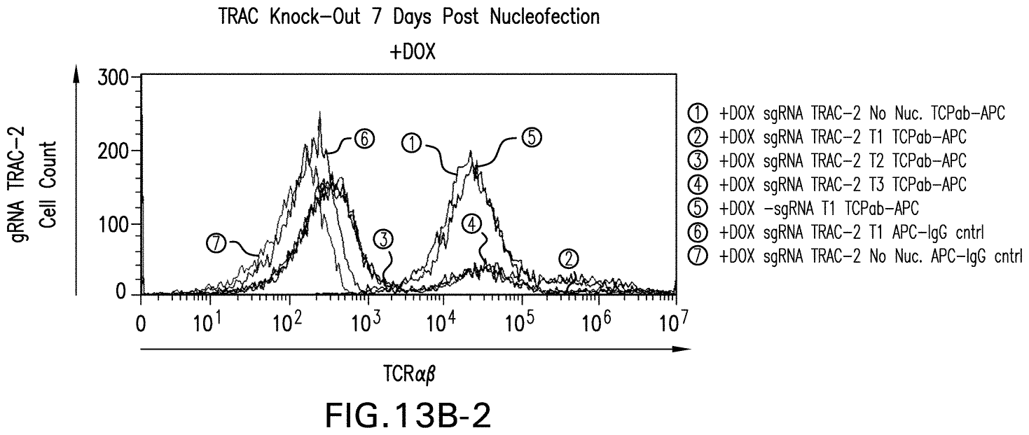

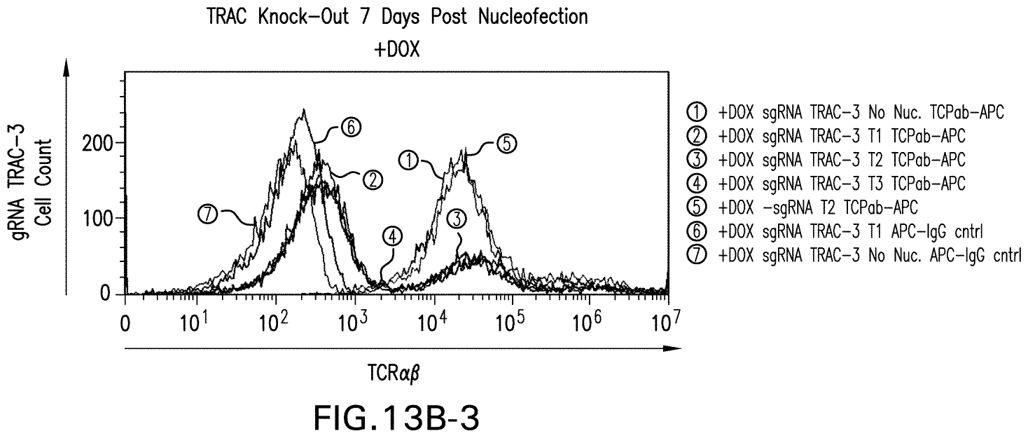

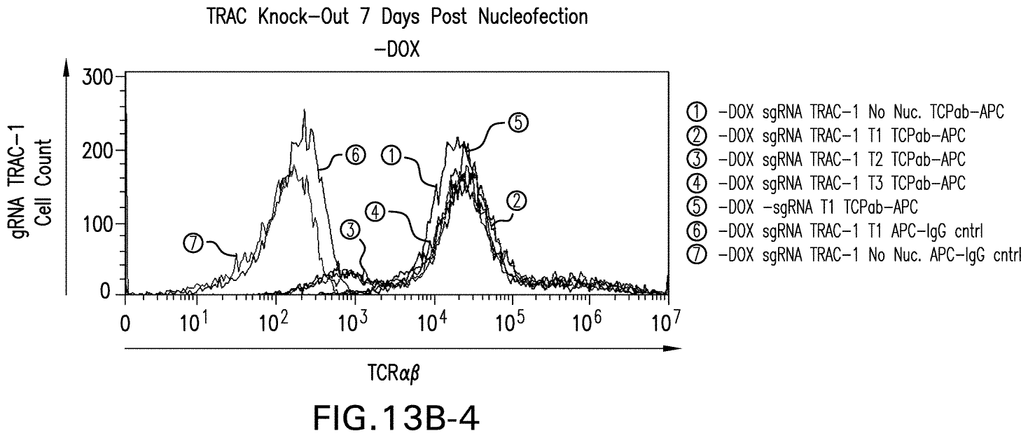

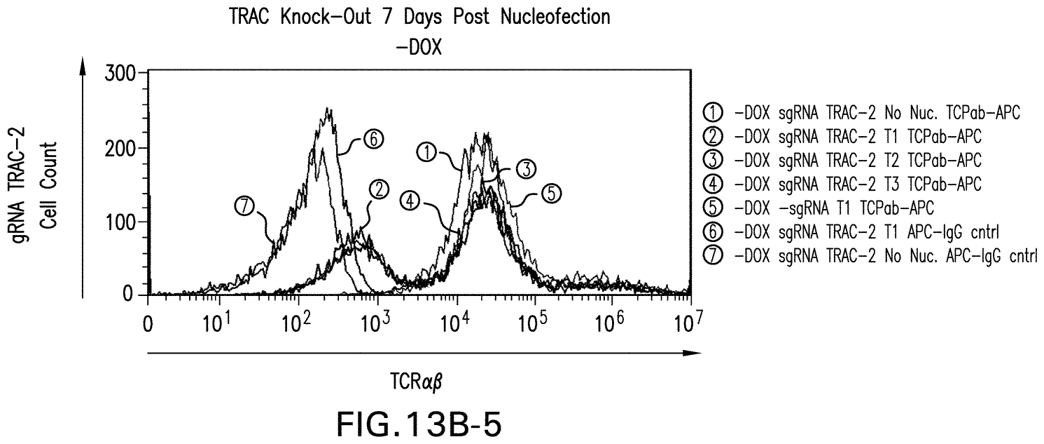

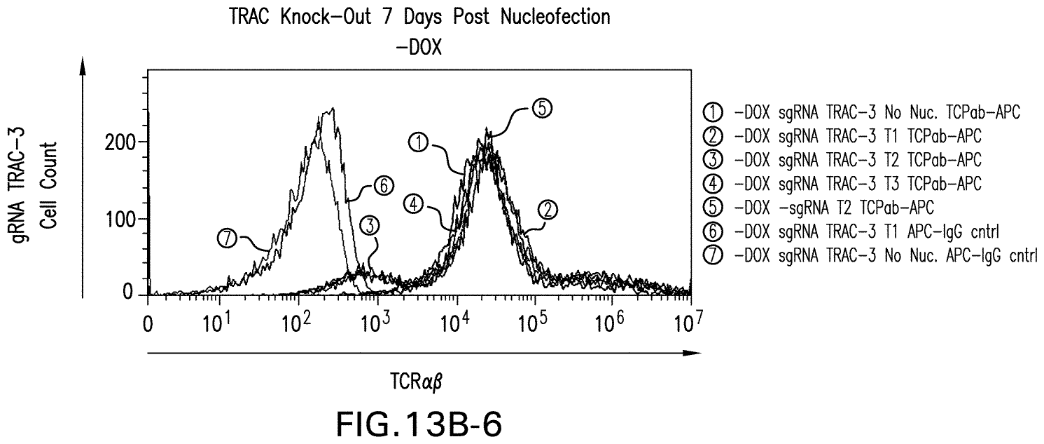

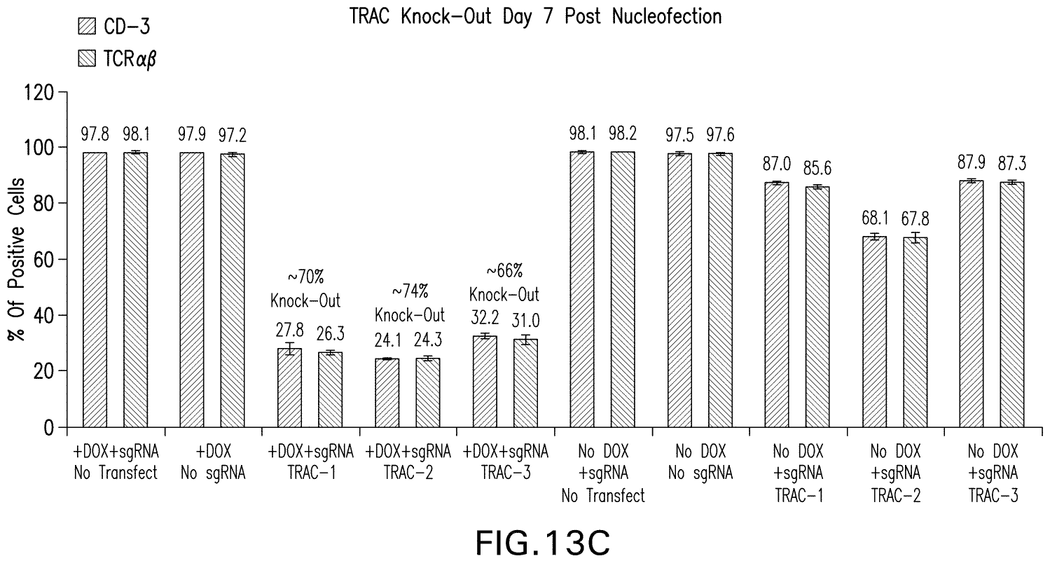

FIGS. 13A-1 through 13B-6 show TRAC knock-out 7 days post nucleofection, with TRAC #1-#3 sgRNA sequences. FIG. 13C shows a compilation of the results. As indicated, each of TRAC #1-TRAC #3 resulted in about 66-74% knock out of the TRAC gene, with TRAC #2 sgRNA showing the highest amount of knock-out (74%).

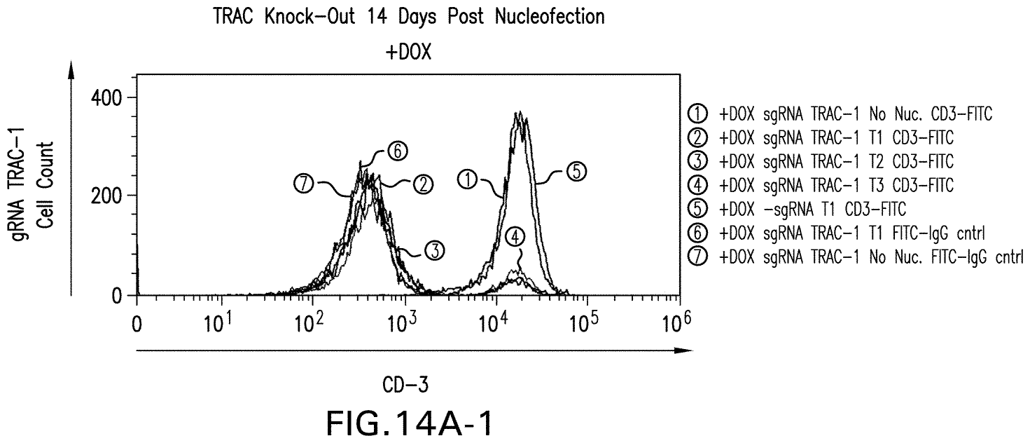

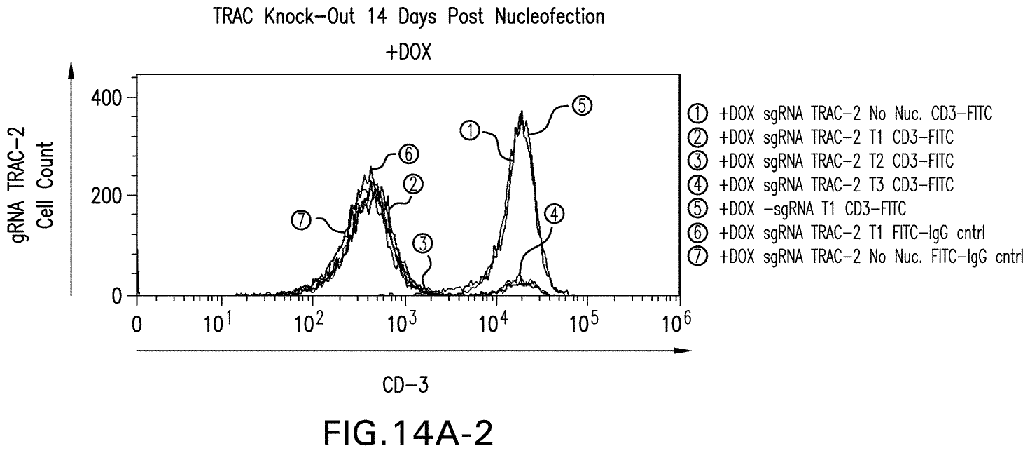

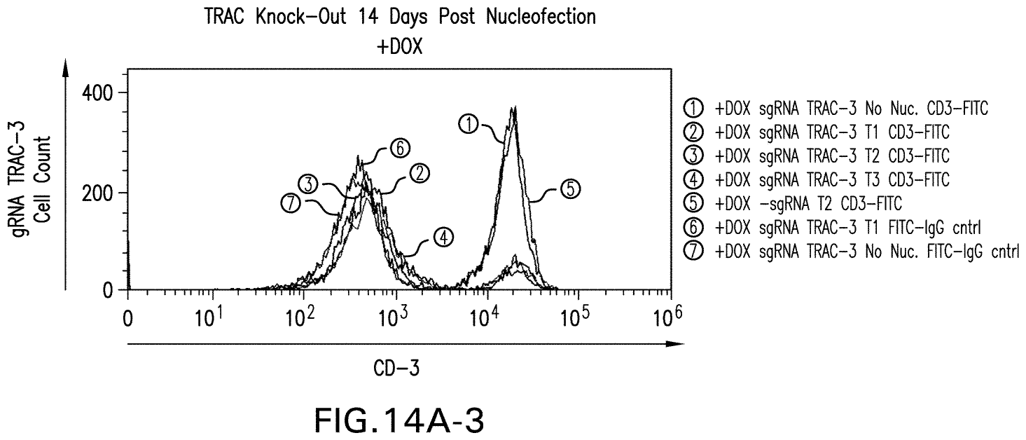

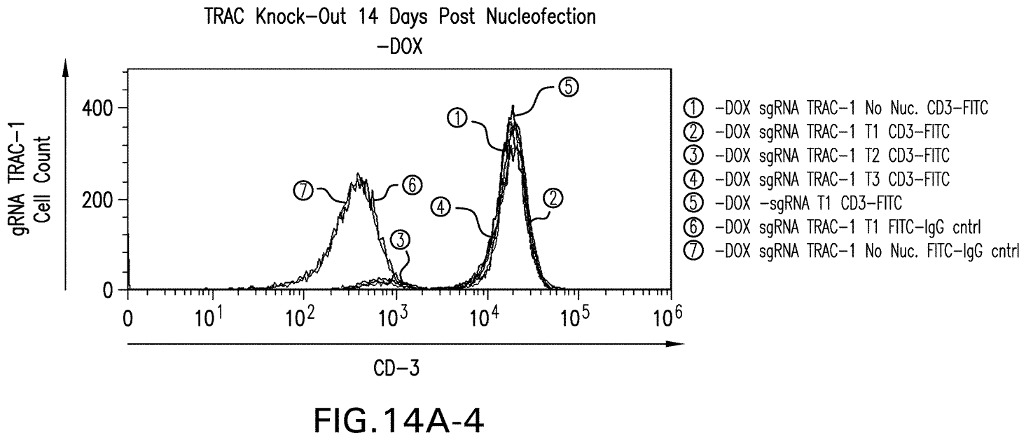

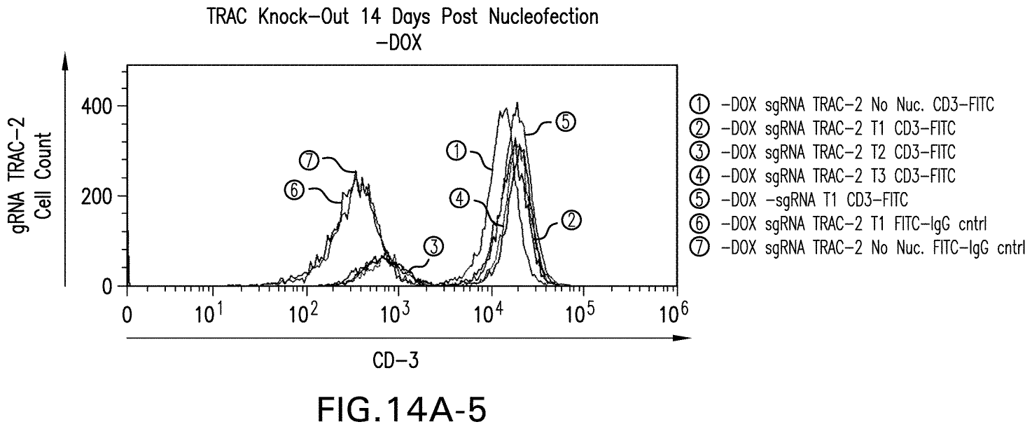

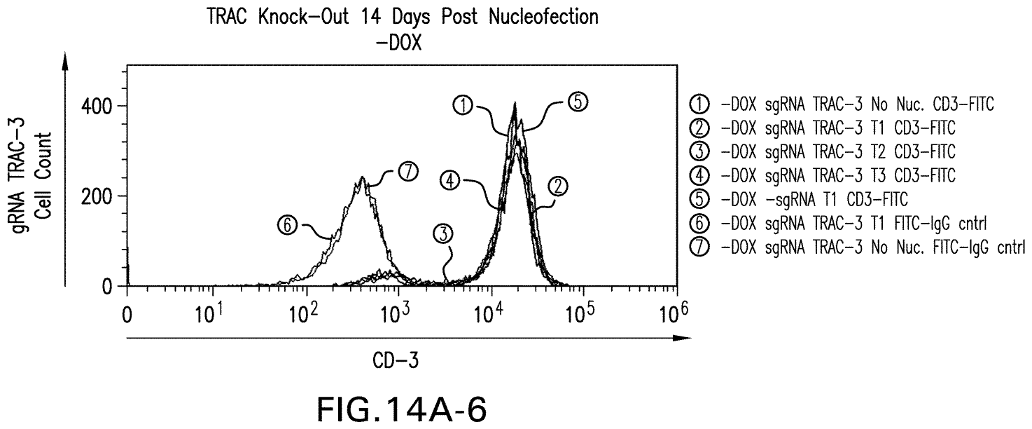

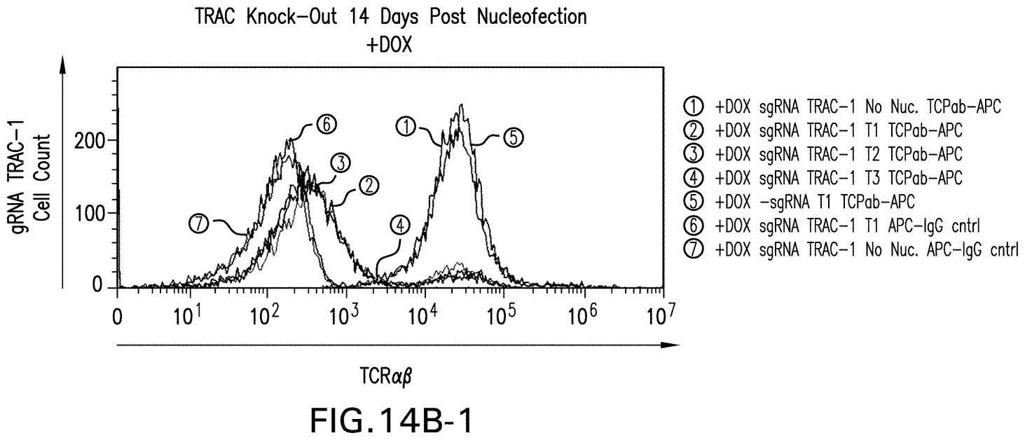

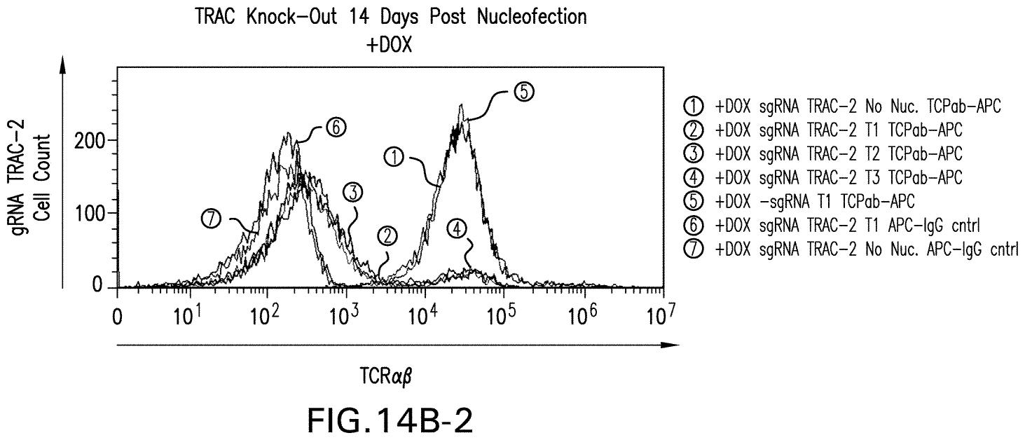

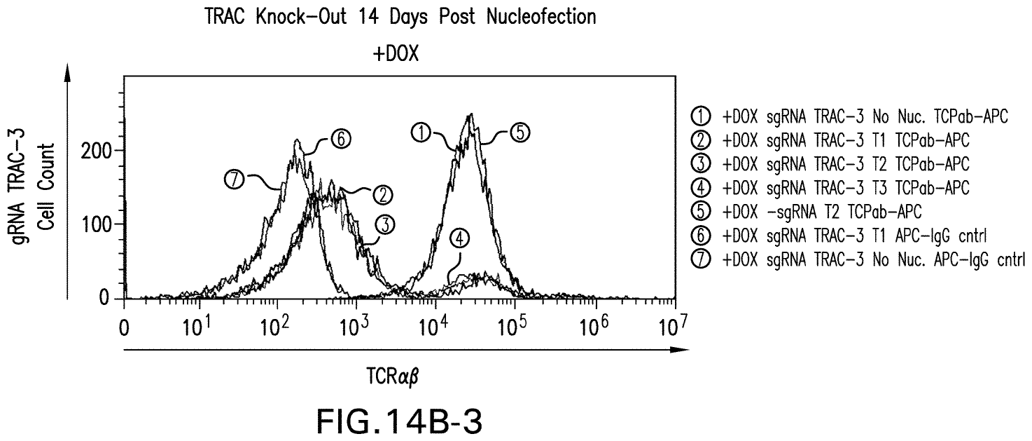

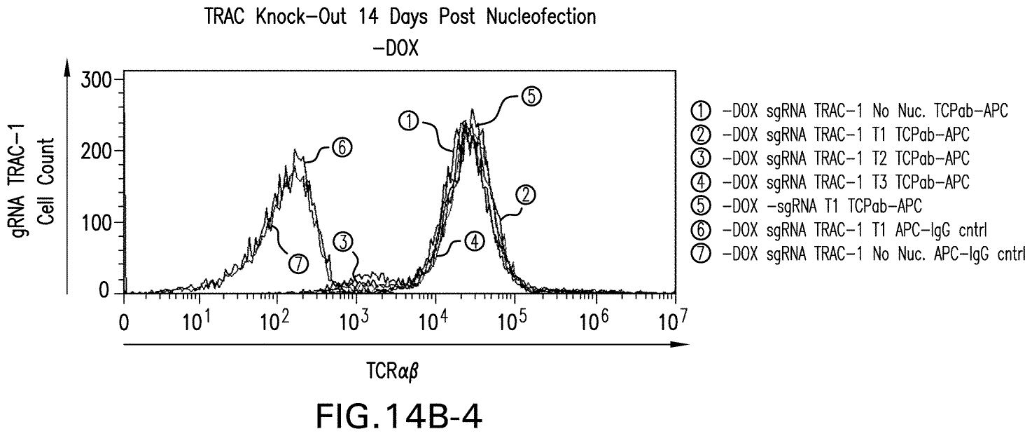

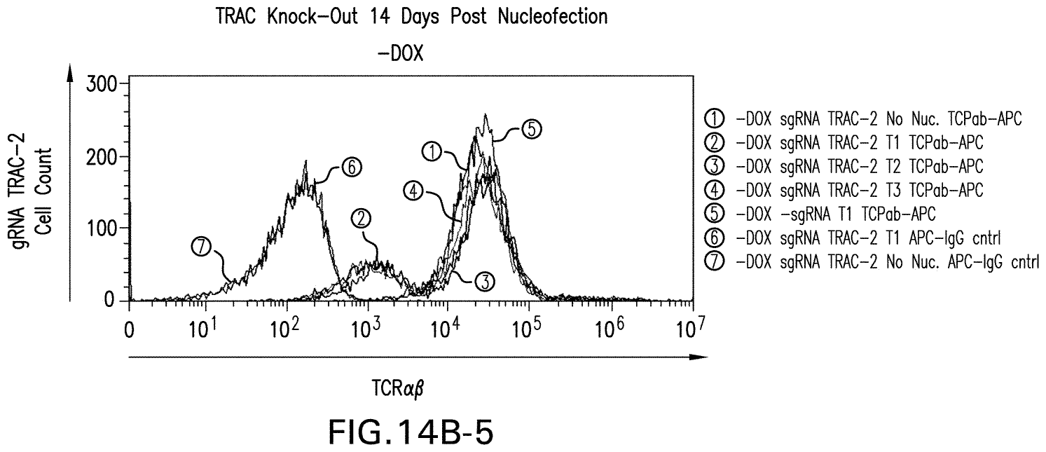

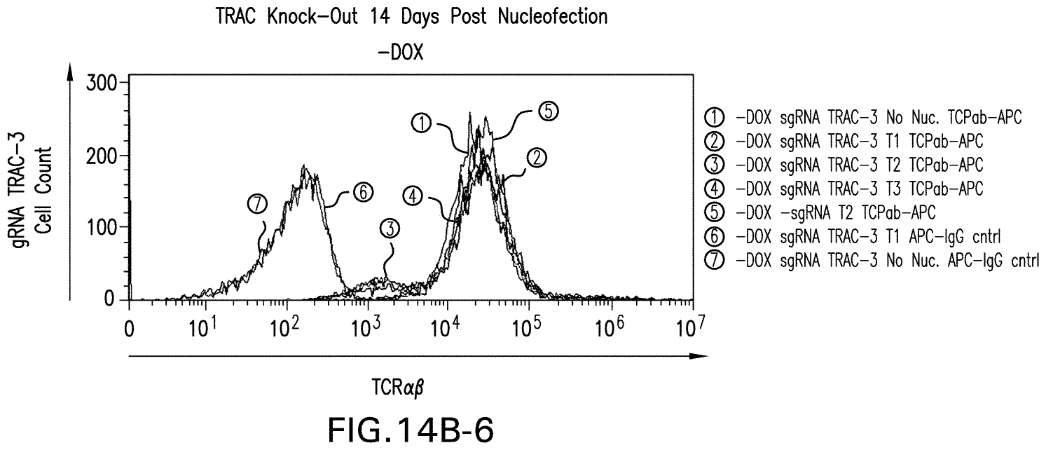

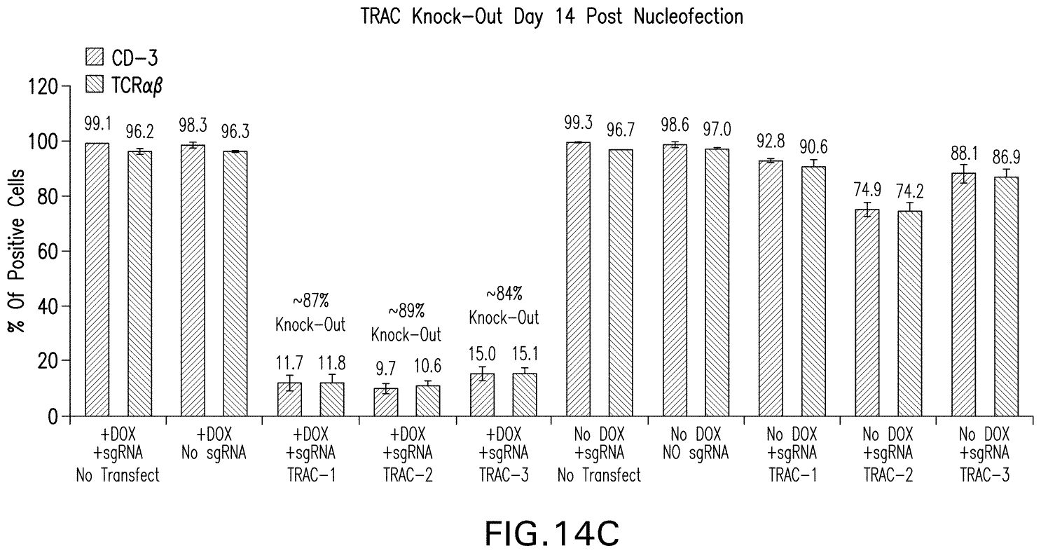

FIGS. 14A-1 through 14B-6 show TRAC knock-out 14 days post nucleofection, with TRAC #1-#3 sgRNA sequences. FIG. 14C shows a compilation of the results. As indicated, each of TRAC #1-TRAC #3 resulted in about 84-89% knock out of the TRAC gene, with TRAC #2 sgRNA showing the highest amount of knock-out (89%).

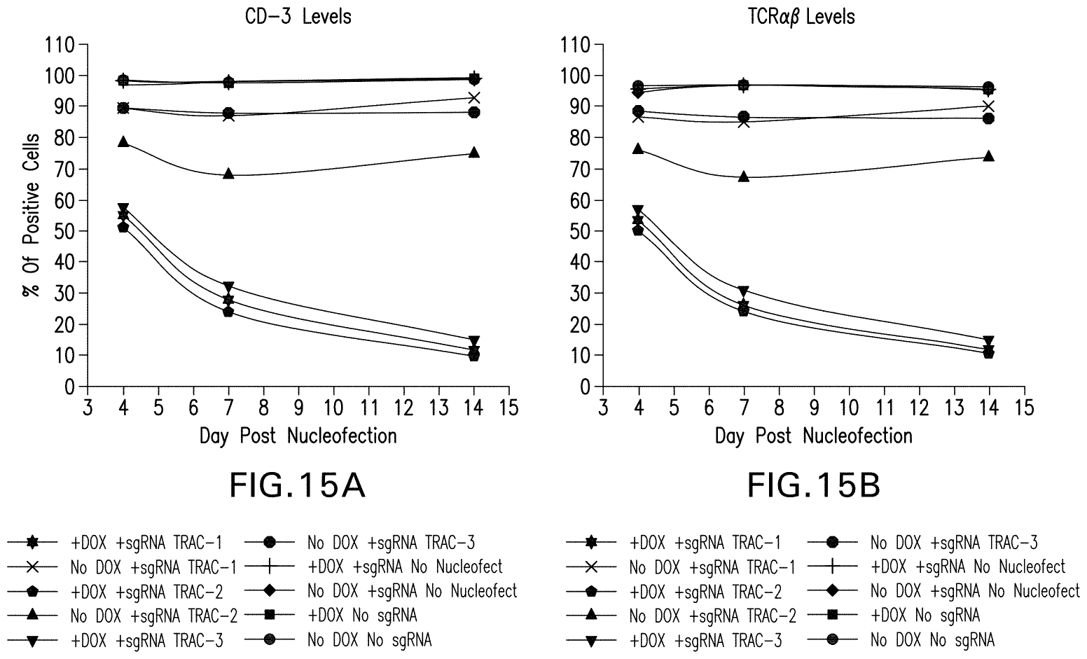

A compilation of the 14 days of experiments are shown in FIGS. 15A-15B, illustrating the effective knock-out of the TRAC gene using the methods described herein.

### Example 3: Knock-in of CAR Construct

The following experiments are designed to demonstrate the ability to knock-in a desired CAR constructed into the Cas9 containing T-Cells.

CAR knock-in experiments are to be conducted following the knock-out experiment described above with the following additions. During the nucleofection of the gRNA (towards the TRAC gene) the addition of a DNA template is performed. This DNA template is designed to integrate into the TRAC locus at the sight of the double-strand break generated by the nuclease using a homologous recombination mechanism.

Three types of DNA templates are to be examined:

single strand DNA

Mini circle DNA

Linear double stranded DNA

Two constructs are to be examined:

Regular anti-CD19 CAR to be evaluated using FACS or functional assay

Anti CD19-CAR fused to a green fluorescent protein (GFP) molecule at the cytosolic side which can be easily detected using FACS.

### Additional Exemplary Embodiments

Embodiment 1 is a method of producing a T-cell line for use in an allogeneic application, comprising: introducing a nucleic acid molecule encoding an engineered nuclease under the control of a controllable promoter into a T-cell line; integrating the nucleic acid molecule into the genome of the T-cell line; and expanding the T-cell line.

Embodiment 2 includes the method of embodiment 1, wherein the engineered nuclease is selected from the group consisting of a meganuclease, a zinc finger nuclease, a transcription activator-like effector-based nuclease, and a CRISPR-associated nuclease.

Embodiment 3 includes the method of embodiment 2, wherein the CRISPR-associated nuclease is a Cas9 nuclease or a Cas12 nuclease.

Embodiment 4 includes the method of any one of embodiments 1-3, wherein the controllable promoter is an inducible promoter.

Embodiment 5 includes the method of embodiment 4, wherein the inducible promoter is a 4HT inducible promoter, a rapamycin inducible promoter, a hormone response element, or a glutamate inducible promoter.

Embodiment 6 includes the method of any one of embodiments 1-3, wherein the controllable promoter is a derepressible promoter.

Embodiment 7 includes the method of embodiment 6, wherein the derepressible promoter includes one or more tetracycline operator sequences (TetO).

Embodiment 8 includes the method of embodiment 7, wherein the nucleic acid molecule further includes a tetracycline repressor protein.

Embodiment 9 includes the method of any one of claims 1-3, wherein the controllable promoter comprises a Tet-on system.

Embodiment 10 includes the method of any one of embodiments 1-9, further comprising freezing the expanded T-cell line following the expanding.

Embodiment 11 includes the method of any one of embodiments 1-10, wherein the nucleic acid molecule is introduced into the T-cell line using a lentiviral vector.

Embodiment 12 includes the method of any one of embodiments 1-11, wherein the T-cell line comprises at least about 10⁹ T-cells.

Embodiment 13 is a method of producing a genetically modified T-cell line, comprising: introducing a nucleic acid molecule encoding a CRISPR-associated nuclease under the control of a controllable promoter into a T-cell line; integrating the nucleic acid molecule into the genome of the T-cell line; expanding the T-cell line; inducing expression of the CRISPR-associated nuclease by activating the controllable promoter; introducing a guide-RNA and a gene of interest into the expanded T-cell line; knocking out expression of a T-cell receptor and introducing the gene of interest into the genome of the T-cell line; and recovering the genetically modified T-cell line.

Embodiment 14 includes the method of embodiment 13, wherein the CRISPR-associated nuclease is a Cas9 nuclease or a Cas12 nuclease.

Embodiment 15 includes the method of embodiment 13 or embodiment 14, wherein the controllable promoter is an inducible promoter.

Embodiment 16 includes the method of embodiment 15, wherein the inducible promoter is a 4HT inducible promoter or a glutamate inducible promoter.

Embodiment 17 includes the method of embodiment 13 or embodiment 14, wherein the controllable promoter is a derepressible promoter.

Embodiment 18 includes the method of embodiment 17, wherein the derepressible promoter includes one or more tetracycline operator sequences (TetO).

Embodiment 19 includes the method of embodiment 18, wherein the nucleic acid molecule further includes a tetracycline repressor protein.

Embodiment 20 includes the method of embodiment 18 or embodiment 19, wherein the activating the derepressible promoter comprises adding doxycycline to the T-cell line.

Embodiment 21 includes the method of embodiment 13 or 14, wherein the controllable promoter comprises a Tet-on system.

Embodiment 22 includes the method of embodiment 21, wherein the activating the Tet-on system comprises added doxycycline to the T-cell line.

Embodiment 23 includes the method of any one of embodiments 13-22, wherein the gene of interest encodes a chimeric antigen receptor (CAR).

Embodiment 24 includes the method of any one of embodiments 13-23, further comprising freezing the T-cell line following the expanding in c, and thawing prior to the inducing in d.

Embodiment 25 includes the method of any one of embodiments 13-24, wherein the genetically modified T-cell line comprises at least about 10⁹ T-cells.

Embodiment 26 is a method of producing a chimeric antigen receptor (CAR) T-cell line, comprising: introducing a nucleic acid molecule encoding a Cas9 nuclease under the control of a controllable promoter into a T-cell line; integrating the nucleic acid molecule into the genome of the T-cell line; expanding the T-cell line; inducing expression of the Cas9 nuclease by activating the controllable promoter; introducing a guide-RNA and a nucleic acid encoding a chimeric antigen receptor (CAR) into the expanded T-cell line; knocking out expression of a T-cell receptor and introducing the nucleic acid encoding the CAR into the genome of the T-cell line; and recovering the CAR T-cell line.

Embodiment 27 includes the method of embodiment 26, wherein the controllable promoter is an inducible promoter.

Embodiment 28 includes the method of embodiment 27, wherein the inducible promoter is a 4HT inducible promoter or a glutamate inducible promoter.

Embodiment 29 includes the method of embodiment 26, wherein the controllable promoter is a derepressible promoter.

Embodiment 30 includes the method of embodiment 29 wherein the derepressible promoter includes one or more tetracycline operator sequences (TetO).

Embodiment 31 includes the method of embodiment 30, wherein the nucleic acid molecule further includes a tetracycline repressor protein.

Embodiment 32 includes the method of embodiment 30 or embodiment 31, wherein the activating the derepressible promoter comprises adding doxycycline to the T-cell line.

Embodiment 33 includes the method of embodiment 26, wherein the controllable promoter comprises a Tet-on system.

Embodiment 34 includes the method of embodiment 33, wherein the Tet-on system comprises adding doxycycline to the T-cell line.

Embodiment 35 includes the method of any one of embodiments 26-34, further comprising freezing the T-cell line following the expanding in c, and thawing prior to the inducing in d.

Embodiment 36 includes the method of any one of embodiments 26-35, wherein the CAR T-cell line comprises at least about 10⁹ T-cells.

Embodiment 37 is an allogeneic T-cell line, comprising a CRISPR-associated (Cas) nuclease under the control of a controllable promoter integrated into the genome of the T-cell line.

Embodiment 38 includes the allogeneic T-cell line of embodiment 37, wherein the CRISPR-associated nuclease is a Cas9 nuclease or a Cas12 nuclease.

Embodiment 39 includes the allogeneic T-cell line of embodiment 37 or embodiment 38, wherein the controllable promoter is an inducible promoter.

Embodiment 40 includes the allogeneic T-cell line of embodiment 39, wherein the inducible promoter is a 4HT inducible promoter or a glutamate inducible promoter.

Embodiment 41 includes the allogeneic T-cell line of embodiment 37 or embodiment 38, wherein the controllable promoter is a derepressible promoter.

Embodiment 42 includes the allogeneic T-cell line of embodiment 41, wherein the derepressible promoter includes one or more tetracycline operator sequences (TetO).

Embodiment 43 includes the allogeneic T-cell line of embodiment 42, wherein T-cell further includes a nucleic acid encoding a tetracycline repressor protein.

Embodiment 44 includes the allogeneic T-cell line of embodiment 37 or embodiment 38, wherein the controllable promoter comprises a Tet-on system.

Embodiment 45 includes the allogeneic T-cell line of any one of embodiments 37-44, comprising at least about 10⁹ T-cells.

Embodiment 46 includes the allogeneic T-cell line of embodiment 45, comprising at least about 10¹⁰ T-cells.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A method of producing a T-cell line for use in an allogeneic application, comprising:
a. introducing a nucleic acid molecule encoding an engineered nuclease under the control of a controllable promoter into a T-cell line;
b. integrating the nucleic acid molecule into the genome of the T-cell line; and
c. expanding the T-cell line.

2. The method of claim 1, wherein the engineered nuclease is selected from the group consisting of a meganuclease, a zinc finger nuclease, a transcription activator-like effector-based nuclease, and a CRISPR-associated nuclease.

3. The method of claim 2, wherein the CRISPR-associated nuclease is a Cas9 nuclease or a Cas12 nuclease.

4. The method of claim 1, wherein the controllable promoter is an inducible promoter, selected from the group consisting of a 4HT inducible promoter, a rapamycin inducible promoter, a hormone response element, and a glutamate inducible promoter.

5. (canceled)

6. The method of claim 1, wherein the controllable promoter comprises a Tet-on system and is a derepressible promoter, and wherein the derepressible promoter includes one or more tetracycline operator sequences (TetO).

7. (canceled)

8. The method of claim 6, wherein the nucleic acid molecule further includes a tetracycline repressor protein.

9. (canceled)

10. The method of claim 1, wherein the T-cell line comprises at least about 10⁹ T-cells and following the expanding, the method further comprises freezing the expanded T-cell line.

11. (canceled)

12. (canceled)

13. A method of producing a genetically modified T-cell line, comprising:
a. introducing a nucleic acid molecule encoding a CRISPR-associated nuclease under the control of a controllable promoter into a T-cell line;
b. integrating the nucleic acid molecule into the genome of the T-cell line;
c. expanding the T-cell line;
d. inducing expression of the CRISPR-associated nuclease by activating the controllable promoter;
e. introducing a guide-RNA and a gene of interest into the expanded T-cell line;
f. knocking out expression of a T-cell receptor and introducing the gene of interest into the genome of the T-cell line; and
g. recovering the genetically modified T-cell line.

14. The method of claim 13, wherein the CRISPR-associated nuclease is a Cas9 nuclease or a Cas12 nuclease.

15. The method of claim 13, wherein the controllable promoter is an inducible promoter selected from the group consisting of a 4HT inducible promoter and a glutamate inducible promoter.

16. (canceled)

17. The method of claim 13, wherein the controllable promoter is a derepressible promoter, and wherein the derepressible promoter includes one or more tetracycline operator sequences (TetO) and activating the derepressible promoter comprises adding doxycycline to the T-cell line.

18. (canceled)

19. The method of claim 17, wherein the nucleic acid molecule further includes a tetracycline repressor protein.

20. (canceled)

21. The method of claim 13, wherein the controllable promoter comprises a Tet-on system, and wherein the activating the Tet-on system comprises adding doxycycline to the T-cell line.

22. (canceled)

23. The method of claim 13, wherein the gene of interest encodes a chimeric antigen receptor (CAR).

24. The method of claim 13, wherein the genetically modified T-cell line comprises at least about 10⁹ T-cells and following expanding of the T-cells, and wherein the method further comprises freezing the T-cell line and thawing prior to the inducing.

25. (canceled)

26. A method of producing a chimeric antigen receptor (CAR) T-cell line, comprising:
a. introducing a nucleic acid molecule encoding a Cas9 nuclease or a Cas12 nuclease under the control of a controllable promoter into a T-cell line;
b. integrating the nucleic acid molecule into the genome of the T-cell line;
c. expanding the T-cell line;
d. inducing expression of the Cas9 nuclease or Cas12 nuclease by activating the controllable promoter;
e. introducing a guide-RNA and a nucleic acid encoding a chimeric antigen receptor (CAR) into the expanded T-cell line;
f. knocking out expression of a T-cell receptor and introducing the nucleic acid encoding the CAR into the genome of the T-cell line; and
g. recovering the CAR T-cell line.

27. The method of claim 26, wherein the controllable promoter is an inducible promoter selected from the group consisting of a 4HT inducible promoter and a glutamate inducible promoter.

28. (canceled)

29. The method of claim 26, wherein the controllable promoter is a derepressible promoter, and wherein the derepressible promoter includes one or more tetracycline operator sequences (TetO) and activating the derepressible promoter comprises adding doxycycline to the T-cell line.

30. (canceled)

31. The method of claim 29, wherein the nucleic acid molecule further includes a tetracycline repressor protein.

32. (canceled)

33. The method of claim 26, wherein the controllable promoter comprises a Tet-on system, and wherein the activating the Tet-on system comprises adding doxycycline to the T-cell line.

34. (canceled)

35. The method of claim 26, further comprising freezing the T-cell line following the expanding in c, and thawing prior to the inducing in d.

36. The method of claim 26, wherein the CAR T-cell line comprises at least about 10⁹ T-cells.

37. An allogeneic T-cell line, comprising a CRISPR-associated (Cas) nuclease under the control of a controllable promoter integrated into the genome of the T-cell line.

38. The allogeneic T-cell line of claim 37, wherein the CRISPR-associated nuclease is a Cas9 nuclease or a Cas12 nuclease.

39. The allogeneic T-cell line of claim 37, wherein the controllable promoter is an inducible promoter selected from the group consisting of a 4HT inducible promoter and a glutamate inducible promoter.

40. (canceled)

41. The allogeneic T-cell line of claim 37, wherein the controllable promoter is a derepressible promoter, and wherein the derepressible promoter includes one or more tetracycline operator sequences (TetO).

42. (canceled)

43. The allogeneic T-cell line of claim 41, wherein T-cell further includes a nucleic acid encoding a tetracycline repressor protein.

44. The allogeneic T-cell line of claim 37, comprising at least about 10⁹ T-cells and, wherein the controllable promoter comprises a Tet-on system.

45. (canceled)

46. The allogeneic T-cell line of claim 44, comprising at least about 10¹⁰ T-cells.

47. The allogeneic T-cell line of claim 44, further comprising a transposon.

48. The method of claim 1, wherein the integrating the nucleic acid molecule into the genome of the T-cell line includes the use of a transposon.

49. The method of claim 13, wherein the integrating the nucleic acid molecule into the genome of the T-cell line includes the use of a transposon.

50. The method of claim 26, wherein the integrating the nucleic acid molecule into the genome of the T-cell line includes the use of a transposon.


SEQUENCE LISTING (4 sequences)

SEQ ID NO: 1 - RNA, 20 nt, Artificial Sequence
  Description of Artificial Sequence Synthetic oligonucleotide
ucucucagcugguacacggc

SEQ ID NO: 2 - RNA, 20 nt, Artificial Sequence
  Description of Artificial Sequence Synthetic oligonucleotide
cucucagcugguacacggca

SEQ ID NO: 3 - RNA, 20 nt, Artificial Sequence
  Description of Artificial Sequence Synthetic oligonucleotide
gagaaucaaaaucggugaau

SEQ ID NO: 4 - protein, 32 aa, Artificial Sequence
  Description of Artificial Sequence Synthetic polypeptide
  MOD_RES (1)..(1): Any amino acid
XIQNPDPAVYQLRDSKSSDKSVCLFTDFDSQT